(19) United States
(12) Patent Application Publication
(10) Pub. No.: US 2012/0276098 A1
(43) Pub. Date: Nov. 1, 2012

(54) DRUG FUSIONS AND CONJUGATES WITH EXTENDED HALF LIFE

(76) Inventors: Bruce Hamilton, Cambridge (GB); Christopher Herring, Cambridge (GB); Mark Andrew Paulik, Durham, NC (US)

(21) Appl. No.: 13/498,924

(22) PCT Filed: Sep. 23, 2010
(86) PCT No.: PCT/EP2010/064020

§ 371 Date: Mar. 29, 2012

Related U.S. Application Data
(60) Provisional application No. 61/247,346, filed on Sep. 30, 2009.

(51) Int. Cl. C07K 19/00; C12N 15/62; A61P 3/04; A61P 3/10; A61P 3/08; A61K 39/395; A61P 3/00

(52) U.S. Cl. 424/134.1; 530/387.3; 536/23.4; 530/391.7; 424/178.1; 530/391.9

(57) ABSTRACT

The present invention relates to drug fusions and conjugates that have improved serum half lives. These fusions and conjugates comprise immunoglobulin (antibody) single variable domains and insulinotropic and/or incretin and/or gut peptide molecules. The invention further relates to uses, formulations, compositions and devices comprising such drug fusions and conjugates. The invention also relates to compositions which comprise more than one insulinotropic and/or incretin and/or gut peptide molecules present as part of a fusion or conjugate and to uses and formulations thereof.

The present invention relates to drug fusions and conjugates that have improved serum half lives. These fusions and conjugates comprise immunoglobulin (antibody) single variable domains and insulinotropic and/or incretin and/or gut peptide molecules. The invention further relates to uses, formulations, compositions and devices comprising such drug fusions and conjugates. The invention also relates to compositions which comprise more than one insulinotropic and/or incretin and/or gut peptide molecules present as part of a fusion or conjugate and to uses and formulations thereof.

### BACKGROUND OF THE INVENTION

Many drugs that possess activities that could be useful for therapeutic and/or diagnostic purposes have limited value because they are rapidly eliminated from the body when administered. For example, many polypeptides that have therapeutically useful activities are rapidly cleared from the circulation via the kidney. Accordingly, a large dose must be administered in order to achieve a desired therapeutic effect or frequent dosing regimen. A need exists for improved therapeutic and diagnostic agents that have improved pharmacokinetic properties.

One such class of drugs that have a short half life in the body or systemic circulation is the incretin hormones such as Glucagon-like peptide 1, and also exendin, for example exendin-4, and other gut peptides such as PYY.

Glucagon-like peptide (GLP)-1 is an incretin hormone with potent glucose-dependent insulinotropic and glucagonostatic actions, trophic effects on the pancreatic β cells, and inhibitory effects on gastrointestinal secretion and motility, which combine to lower plasma glucose and reduce glycemic excursions. Furthermore, via its ability to enhance satiety, GLP-1 reduces food intake, thereby limiting weight gain, and may even cause weight loss (Drucker (2002) Gastroenterology 122:531-544, Giorgiano et al. (2006) Diabetes Research and Clinical Practice 74:S152-155), Holt (2002) Diabetes/Metabolism Research and Reviews 18:430-441. Taken together, these actions give GLP-1 a unique profile, considered highly desirable for an antidiabetic agent, particularly since the glucose dependency of its antihyperglycemic effects should minimize any risk of severe hypoglycemia. However, its pharmacokinetic/pharmacodynamic profile is such that native GLP-1 is not therapeutically useful. Thus, while GLP-1 is most effective when administered continuously, single subcutaneous injections have short-lasting effects. GLP-1 is highly susceptible to enzymatic degradation in vivo, and cleavage by dipeptidyl peptidase IV (DPP-IV) is probably the most relevant, since this occurs rapidly and generates a non insulinotropic metabolite (Metlein (1999) Regulatory Peptides 85:9-244). Strategies for harnessing GLP-1's therapeutic potential, based on an understanding of factors influencing its metabolic stability and pharmacokinetic/pharmacodynamic profile, have therefore been the focus of intense research.

Extensive work has been done to attempt to inhibit the peptidase or to modify GLP-1 in such a way that its degradation is slowed down while still maintaining biological activity. WO05/027978 discloses GLP-1 derivatives having a protracted profile of action. WO 02/46227 discloses heterologous fusion proteins comprising a polypeptide (for example, albumin) fused to GLP-1 or analogues (the disclosure of these analogues is incorporated herein by reference as examples of GLP-1 analogues that can be used in the present invention). WO05/003296, WO03/060071, WO03/059934 disclose amino fusion protein wherein GLP-1 has fused with albumin to attempt to increase the half-life of the hormone.

Peptide YY is a short (36 amino acid) protein released by neuroendocrine cells in response to feeding. PYY concentration in the circulation increases postprandially and decreases on fasting. It exerts its action through NPY receptors, inhibiting gastric motility and increasing water and electrolyte absorption in the colon. It is secreted by the neuroendocrine cells in the ileum and colon in response to a meal, and has been shown to reduce appetite Ballantyne (2006) Obesity Surgery 16:651-658, Batterham (2003) New England Journal of Medicine 349:941-8, Boey et al. (2007) Peptides 28:390-395, and Karra et al. (2009) Journal of Physiology 587:19-25).

Exendin-4 is a hormone found in the saliva of the Gila monster it is an agonist of GLP-1 and also has a very potent insulinotropic effects. In contrast to GLP-1, exendin-4 has a much longer in vivo half-life It displays biological properties similar to human glucagon-like peptide-1 (GLP-1) in its regulation of glucose metabolism and insulin secretion. Exendin-4 enhances glucose-dependent insulin secretion by the pancreatic beta-cell, suppresses inappropriately elevated glucagon secretion, and slows gastric emptying. (DeFronzo et al. (2005) Diabetes Care 28:5:1092-100, Edwards et al. (2001) American Journal of Physiology: Endocrinology and Metabolism 281:E155-162, Kolterman et al. (2003) Journal of Clinical Endocrinology and Metabolism 88(7):3082-9, and Nielsen et al. (2004) Regulatory Peptides 117:77-88).

In medicine, there remains a tremendous need for improved compositions comprising incretins and/or insulinotropic and/or gut peptide agents such as GLP-1 peptides, PYY, exendin, or other agents that have an insulinotropic and/or incretin effect /or anorexic effect and which can be used in medicine e.g. in the treatment and/or prevention of metabolic conditions such as diabetes and obesity.

There is thus a need to provide new therapeutic compositions comprising incretins/insulinotropic/gut peptide containing agents (e.g. GLP-1, exendin -4, PYY,) to provide more potent and longer duration of action in vivo while maintaining their low toxicity and therapeutic advantages.

### SUMMARY OF THE INVENTION

The present invention thus provides (a) compositions which comprise (or consist of) a single molecule (e.g. a single fusion or conjugate) which comprises combinations of (i.e. two or more) molecules selected from incretins and/or insulinotropic agents and/or gut peptides, which are e.g. present as fusions (chemical or genetic) or as conjugates; or alternatively (b) a composition which comprises two or more individual molecules wherein each individual molecule comprises one or more incretins and/or insulinotropic agents and/or gut peptides. These compositions (a) and/or (b) can also comprise further proteins or polypeptides e.g. half life extending proteins or polypeptides or peptides e.g. which can bind to serum albumin for example to human serum albumin e.g. a dAb (a domain antibody) e.g. a dAb which binds to serum albumin such as human serum albumin (Albudab™).

In one embodiment the present invention provides a composition which comprises (or consists of) a single fusion (chemical or genetic) or a single conjugate molecule, wherein said fusion or conjugate comprises or consists of (a) two or more molecules which are selected from: insulinotropic and/or incretin molecules and/or gut peptides, (e.g. a Peptide YY (PYY) peptide, 3-36 PYY, exendin-4, a GLP e.g. a GLP-1 e.g. the GLP-1 (7-37) A8G mutant), which are present as a single fusion or conjugate with (b) a domain antibody (dAb) which binds specifically to serum albumin, (e.g. the DOM 7h-14 (Vk) domain antibody (dAb), (the amino acid sequence of DOM 7h-14 is shown in FIG. 1(h): SEQ ID NO 8), or e.g. the DOM 7h-14 -10(Vk) domain antibody (dAb), (the amino acid sequence of DOM 7h-14-10 is shown in FIG. 1(o): SEQ ID NO 15 , or the DOM 7h-11-15 (the amino acid sequence of DOM 7h-11-15 is shown in FIG. 1(P): SEQ ID NO 16) or e.g. the DOM 7h-14 -10(Vk) domain antibody (dAb) which has the R108C mutation (the amino acid sequence of DOM 7h-14-10 R108C is shown in FIG. 1(r) SEQ ID NO 18) or e.g. the DOM 7h-11 -15(Vk) domain antibody (dAb) or e.g. the DOM 7h-11 -15(Vk) domain antibody (dAb) which has the R108C mutation (the amino acid sequence of DOM 7h-11-15 R108C is shown in FIG. 1(t): SEQ ID NO 47). In one embodiment the fusion or conjugate is not the 2xGLP-1 (7-37) A8G DOM7h-14 dAb fusion (DAT0114, with the amino acid sequence is shown' in FIG. 1 (a): SEQ ID NO 1).

In another embodiment the single fusion or conjugate comprises or consists of a PYY (e.g. PYY 3-36) and an exendin (e.g. exendin-4) and one or more dAbs that bind to serum albumin e.g. human serum albumin e.g. any one of the Albudabs™ described herein. In one embodiment the single fusion has the amino acid sequence shown in FIG. 1 (u): SEQ ID NO 48.

In another embodiment.the present invention further provides compositions which comprise or consist of any of the individual fusions or conjugated molecules described or disclosed herein and their use (e.g. for any of the uses described herein for combinations) when they are administered alone or formulated with any suitable pharmaceutical excipients or additives.

The invention also provides nucleic acids encoding any of the individual fusions described herein:

In one embodiment of the above the incretin/insulinotropic/gut peptide molecules can be different incretin/insulinotropic/gut peptide molecules or they can be the same. The dAb that binds serum albumin (i.e. the AlbudAb™) can also be any one of those described or referenced in for example WO 2006/059106 or WO 05/118642 or WO 2008096158 or PCT/EP2009/053640 or U.S. Ser. No. 61/163,990.

In another embodiment the present invention further provides a composition, which comprises (or consists of) two or more individual fusions or conjugates and wherein each individual fusion or conjugate comprises or consists of (a) one or more molecules selected from: insulinotropic and/or incretin molecules and/or gut peptides, (e.g. a PYY peptide, 3-36 PYY, exendin-4, a GLP e.g. a GLP-1 e.g. the GLP-1 (7-37) A8G mutant), present as a fusion or conjugate with (b) a domain antibody (dAb) which binds specifically to serum albumin (e.g. the DOM 7h-14 (Vk) domain antibody (dAb), (the amino acid sequence of DOM 7h-14 is shown in FIG. 1(h): SEQ ID NO 8) or e.g. the DOM 7h-14 -10(Vk) domain antibody (dAb), (the amino acid sequence of DOM 7h-14-10 is shown in FIG. 1(o): SEQ ID NO 15 , or the DOM 7h-11-15 (the amino acid sequence of DOM 7h-11-15 is shown in FIG. 1(P): SEQ ID NO 16) or e.g. the DOM 7h-14 -10(Vk) domain antibody (dAb) which has the R108C mutation (the amino acid sequence of DOM 7h-14-10 R108 C is shown in FIG. 1(r) SEQ ID NO 18) or e.g. the DOM 7h-11 -15(Vk) domain antibody (dAb) or e.g. DOM 7h-11 -15(Vk) domain antibody (dAb) the which has the R108C mutation (the amino acid sequence of DOM 7h-11-15 R108 C is shown in FIG. 1(t)): SEQ ID NO 47). In one embodiment this composition can comprise one or more molecules selected from those in: FIGS. 1a-1g and FIGS. 1m-1V and also FIG. 3 and also the Dom7h-11-15 (R108C)-PEG-3-36 PYY (Lysine at position 10) molecule (with the structure shown in FIG. 3 except that the AlbudAb component is the Dom7h-11-15 (R108C) AlbudAb.

Such a composition comprising (or consisting of) two or more fusions or conjugates as described above can be a combined preparation for simultaneous, separate or sequential use in therapy, e.g. to treat or prevent a metabolic disease such as hyperglycemia, impaired glucose tolerance, beta cell deficiency, diabetes (for example type 1 or type 2 diabetes or gestational diabetes) non-alcoholic steatotic liver disease, polycystic ovarian syndrome, hyperlipidemia or obesity or diseases characterised by overeating and/or modify energy expenditure.

The fusions or conjugates of the invention can display synergy (by synergy we mean that their effect when administered is more than the simple additive effect of each when administered singly) when administered together or sequentially e.g. as combined combined preparation for simultaneous, separate or sequential use in therapy, e.g to treat or prevent a metabolic disease such as hyperglycemia, impaired glucose tolerance, beta cell deficiency, diabetes (for example type 1 or type 2 diabetes or gestational diabetes) non-alcoholic steatotic live disease, polycystic ovarian syndrome, hyperlipidemia or obesity or diseases characterised by overeating and/or modify energy expenditure.

Synergy can also result from the presence of more than one incretin or insulinotropic or gut peptide on one molecule and also from the interaction between the AlbudAb and the incretin or insulinotropic or gut peptide.

In any one of the compositions according to the invention the incretin and/or insulinotropic molecules and/or gut peptides can be for example selected from: a PYY peptide e.g. 3-36 or 13-36; exendin-4, a GLP e.g. a GLP-1 e.g. the GLP-1 (7-37) A8G mutant, or they can be mutants, analogues or derivatives of these peptides which e.g. can retain incretin/insulinotropic activity. The GLP, PYY, exendin can be any of those described in WO 2006/059106. The mutants, analogues or derivatives of these peptides can be those which retain incretin and/or insulinotropic activity.

The insulinotropic and/or incretin and/or gut peptide molecules (e.g. PYY, exendin, GLP-1, etc) when present as a fusion (or conjugate) with a dAb can be linked to either the N-terminal or C-terminal of the dAb or at points within the dAb sequence. In one embodiment one or more incretin and/or insulinotropic and/or gut peptide molecules are present as a fusion (or conjugate) with the N terminal of the dAb and one or more incretin and/or insulinotropic and/or gut peptide molecules are also present as a fusion (or conjugate) with the C terminal of the dAb.

An amino acid or chemical linker may also optionally be present joining the insulinotropic and/or incretin and/or gut peptide molecules, e.g. exendin-4 and/or GLP-1, e.g. with the dAb. The linker can be for example a helical linker e.g. the helical linker of sequence shown in FIG. 1 (k): SEQ ID NO 11, or it may be a gly-ser linker e.g. with an amino acid sequence shown in FIG. 1 (l): SEQ ID NO 12.

Alternatively the linker can be e.g. a PEG linker e.g. the PEG linker shown in FIG. 3.

In certain embodiments, the fusions (or conjugates) of the invention can comprise further molecules e.g. further peptides or polypeptides.

In one embodiment the invention provides a composition which comprises or consists of the following two individual molecules:
(a) a genetic fusion which is: exendin-4, (G4S)3 linker, 7h-14 AlbudAb (DAT 0115, which has the amino acid sequence present in FIG. 1b: SEQID NO 2); and
(b) a peptide conjugate which is:
a Dom7h-14-10 (R108C) AlbudAb conjugated to a C-terminally amidated PYY3-36 via a lysine (introduced at position 10 of PYY) and a 4 repeat PEG linker. The line represents the linker which is covalently attached to the free C terminal cysteine of the Dom7h-14-10 (R108C) AlbudAb and the lysine at position 10 of the PYY sequence. The amino acid sequence and structure of this peptide conjugate is as follows (and is also shown in FIG. 3):

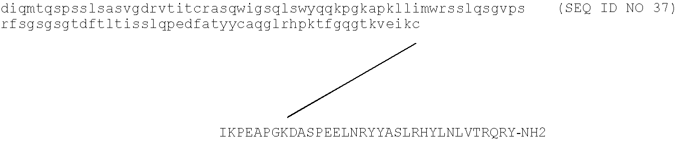

Where the C terminal cysteine of Dom7h-14-10(R108C) is covalently attached to the lysine in the PYY peptide via a linker.

The chemical linker has the following structure:

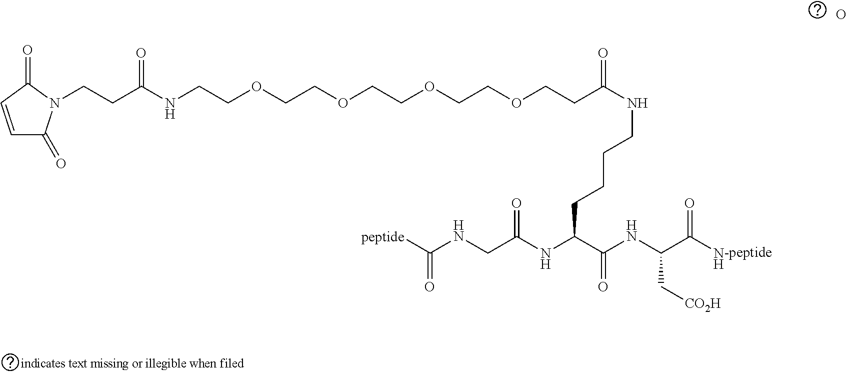

The above two molecules (a) a genetic fusion which is: exendin-4, (G4S)3 linker, 7h-14 AlbudAb (DAT 0115, which has the amino acid sequence present in FIG. 1b) and (b) the peptide conjugate which is:
a Dom7h-14-10 (R108C) AlbudAb conjugated to PYY3-36 via a lysine and 4 repeat PEG linker (of structure shown in FIG. 3) can be present as a combined preparation for simultaneous, separate or sequential suitable for uses in therapy as described herein.

Alternatively in the above composition the peptide conjugate (b) (which is the structure shown in FIG. 3) can be replaced by the following molecule: the Dom7h-11-15 (R108C)-PEG-3-36 PYY (Lysine at position 10) (with the structure shown in FIG. 3 except that the AlbudAb component is the Dom7h-11-15 (R108C).

In yet a further alternative in the above composition the peptide conjugate (b) (which is the structure shown in FIG. 3) can be replaced by the following molecule: the PYY-Dom 7h-14-10 fusion with the amino acid sequence shown in FIG. 1 (v): SEQ ID NO 49.

In a further embodiment the invention provides a composition which comprises or consists of a PYY (e.g. PYY 3-36) and an exendin (e.g. exendin-4) and one or more AlbudAb, e.g. any of the AlbudAbs described herein. In one embodiment the single fusion has the amino acid sequence shown in FIG. 1 (u): SEQ ID NO 48.

Dom 7h-14 is a human immunoglobulin single variable domain or dAb (Vk) that binds to serum albumin and its amino acid sequence is shown in FIG. 1(h): SEQ ID NO 8. The CDR regions of Dom7h-14 dAb are underlined in the amino acid sequence shown in FIG. 1(h): SEQ ID NO 8.

Dom 7h-14-10 is a human immunoglobulin single variable domain or dAb (Vk) that binds to serum albumin and its amino acid sequence is shown in FIG. 1(h): SEQ ID NO 8. The CDR regions of Dom7h-14-10 dAb are underlined in the amino acid sequence shown in FIG. 1(o): SEQ ID NO 15.

Dom 7h-11-15 is a human immunoglobulin single variable domain or dAb (Vk) that binds to serum albumin and its amino acid sequence is shown in FIG. 1(p): SEQ ID NO 16. The CDR regions of Dom7h-11-15 dAb are underlined in the amino acid sequence shown in FIG. 1(p): SEQ ID NO 16.

Dom 7h-14-10 with a R108C mutation is a human immunoglobulin single variable domain or dAb (Vk) that binds to serum albumin and its amino acid sequence is shown in FIG. 1(R): SEQ ID NO 18.

Dom 7h-11-15 with a R108C mutation is a human immunoglobulin single variable domain or dAb (Vk) that binds to serum albumin and its amino acid sequence is shown in FIG. 1(t).

The R108 C mutation refers to a mutation in which the C terminal arginine in the unmutated sequence is replaced by a cysteine and in one aspect of the invention any of the AlbudAbs described herein can have this mutation.

As used herein, “fusion” refers to a fusion protein that comprises as one moiety a dAb that binds serum albumin and further moieties which are insulinotropic and/or incretin and/or gut peptide molecules. The dAb that binds serum albumin and the insulinotropic and/or an incretin and/or gut peptide molecules can be present as discrete parts (moieties) of a single continuous polypeptide chain. The dAb and incretin/insulinotropic/gut peptide moieties can be directly bonded to each other through a peptide bond or linked through a suitable amino acid, or peptide or polypeptide linker. Additional moieties e.g. peptides or polypeptides (e.g. third, fourth) and/or linker sequences, can be present as appropriate. The dAb can be in an N-terminal location, C-terminal location or it can be internal, relative to the incretin/insulinotropic/gut peptide molecules. In certain embodiments the fusion protein contains one or more than one (e.g. one to about 20) dAb moieties.

As used herein, “conjugate” refers to a composition comprising a dAb that binds serum albumin to which an insulinotropic /incretin/gut peptide molecule is covalently or non-covalently bonded. The insulinotropic/incretin/gut peptide molecule can be covalently bonded to the dAb directly or indirectly through a suitable linker moiety. The insulinotropic/incretin/gut peptide molecule can be bonded to the dAb at any suitable position, such as the amino-terminus, the carboxyl-terminus or through suitable amino acid side chains (e.g., the E amino group of lysine, or thiol group of cysteine) either naturally occurring or engineered. Alternatively, the insulinotropic/incretin/gut peptide molecule can be noncovalently bonded to the dAb directly (e.g., electrostatic interaction, hydrophobic interaction) or indirectly (e.g., through noncovalent binding of complementary binding partners (e.g., biotin and avidin), wherein one partner is covalently bonded to insulinotropic/incretin molecule and the complementary binding partner is covalently bonded to the dAb). The dAb can be in an N-terminal location, C-terminal location or it can be internal relative to the incretin/insulinotropic/gut peptide molecules. In certain embodiments the conjugate protein contains one or more than one (e.g. one to about 20) dAb moieties.

The invention also provides compositions comprising nucleic acids encoding the fusions described herein for example comprising nucleic acids shown in FIG. 2.

Also provided are host cells e.g. non-embryonic host cells e.g. prokaryotic or eukaryotic (such as mammalian) hosts cells such as E. coli or yeast host cells that comprise these nucleic acids.

The invention further provides a method for producing a fusion of the present invention which method comprises maintaining a host cell such as those described above that comprises a recombinant nucleic acid and/or construct that encodes a fusion of the invention under conditions suitable for expression of said recombinant nucleic acid, whereby a fusion is produced.

The invention also provides pharmaceutical compositions comprising the compositions of the invention.

The invention further provides a composition of the invention for use in medicine, e.g. for use in the treatment of e.g. a metabolic disease or condition such as hyperglycemia, impaired glucose tolerance, beta cell deficiency, diabetes (for example type 1 or type 2 diabetes or gestational diabetes) non-alcoholic steatotic liver disease, polycystic ovarian syndrome, hyperlipidemia or obesity or diseases characterised by overeating e.g. it can be used to suppress appetite or modify energy expenditure, pancreatitis and also to prevent tumour growth e.g. pancreatic tumour growth (e.g. pancreatic adenocarcinoma) and which comprises administering to said individual a therapeutically effective amount of a composition of the invention.The invention also provides compositions comprising any of the PYY AlbudAb described herein (whether used singly or in combination) for use to treat and/or prevent pancreatitis and also to prevent tumour growth e.g. pancreatic tumour growth (e.g. pancreatic adenocarcinoma).

The invention also provides a method for treating an individual having a disease or disorder, such as those described herein e.g. a metabolic disease or condition such as hyperglycemia, impaired glucose tolerance, beta cell deficiency, diabetes (for example type 1 or type 2 diabetes or gestational diabetes)), non-alcoholic steatotic liver disease, polycystic ovarian syndrome, hyperlipidemia, or obesity or diseases characterised by overeating e.g. it can be used to suppress appetite appetite or modify energy expenditure, pancreatitis and also to prevent tumour growth e.g. pancreatic tumour growth; and which comprises administering to said individual a therapeutically effective amount of a composition of the invention.

Other metabolic diseases or conditions which can be treated or prevented according to the invention include, but are not limited to, insulin resistance, insulin deficiency, hyperinsulinemia, hyperglycemia, dyslipidemia, hyperlipidemia, hyperketonemia, hyperglucagonemia,hypertension, coronary artery disease, atherosclerosis, renal failure, neuropathy (e.g., autonomic neuropathy, parasympathetic neuropathy, and polyneuropathy), retinopathy, cataracts, metabolic disorders (e.g., insulin and/or glucose metabolic disorders), endocrine disorders, obesity, weight loss, liver disorders (e.g., liver disease, steatosis of the liver, cirrhosis of the liver, and disorders associated with liver transplant), and conditions associated with these diseases or disorders.

In addition, conditions associated with diabetes that can be prevented or treated with the compounds of the present invention include, but are not limited to, hyperglycemia, obesity, diabetic retinopathy, mononeuropathy, polyneuropathy, atherosclerosis, ulcers, heart disease, stroke, anemia, gangrene (e.g., of thefeet and hands), impotence, infection, cataract, poor kidney function, malfunctioning of the autonomic nervous system, impaired white blood cell function, Carpal tunnel syndrome, Dupuytren's contracture, and diabetic ketoacidosis.

The invention also provides methods for treating or preventing diseases associated with elevated blood glucose comprising administering at least one dose of a composition e.g. a pharmaceutical composition of the present invention to a patient or subject.

When patient or subject are described in the application this can mean a human or non-human patient or subject.

The invention further relates to methods of regulating insulin responsiveness in a patient, as well as methods of increasing glucose uptake by a cell, and methods of regulating insulin sensitivity of a cell, using the conjugates or fusions of the invention. Also provided are methods of stimulating insulin synthesis and release, enhancing adipose, muscle or liver tissue sensitivity towards insulin uptake, stimulating glucose uptake, slowing digestive process, reducing appetite, modifying energy expenditure,or blocking the secretion of glucagon in a patient, comprising administering to said patient a composition of the invention e.g. comprising administering at least one dose of a composition e.g. a pharmaceutical composition, of the present invention.

The compositions e.g. pharmaceutical compositions, of the invention may be administered alone or in combination with other molecules or moieties e.g. polypeptides, therapeutic proteins (e.g. Albiglutide™ which is two molecules of GLP-1 covalently linked to a molecule of human serum albumin) and/or molecules (e.g., insulin and/or other proteins (including antibodies), peptides, or small molecules that regulate insulin sensitivity, weight, heart disease, hypertension, neuropathy, cell metabolism, and/or glucose, insulin, or other hormone levels, in a patient). In specific embodiments, the conjugates or fusions of the invention are administered in combination with insulin (or an insulin derivative, analog, fusion protein, or secretagogue).

The invention also provides compositions of the invention for use in the treatment of a disease or disorder, such as any of those mentioned above e.g. a metabolic disorder such as hyperglycemia, pancreatitis, diabetes (type 1 or 2 or gestational diabetes) or obesity or diseases characterized by gut hypermotility, and also to prevent tumour growth e.g. pancreatic tumour growth (e.g. pancreatic adenocarcinoma).

The invention also provides for use of a composition of the invention in the manufacture of a medicament for treatment of a disease or disorder, such as any of those mentioned above e.g. a metabolic disorder such as hyperglycemia, diabetes (type 1 or 2 or gestational diabetes) or obesity, pancreatitis, or diseases characterized by gut hypermotility and also e.g. pancreatic tumour growth (e.g. pancreatic adenocarcinoma).

The invention also relates to use of any of the compositions described herein for use in therapy, diagnosis or prophylaxis.

The compositions of the invention, e.g. the dAb component of the composition, can be further formatted to have a larger hydrodynamic size to further extend the half life, for example, by attachment of a PEG group, serum albumin, transferrin, transferrin receptor or at least the transferrin-binding portion thereof, an antibody Fc region, or by conjugation to an antibody domain. For example, the dAb that binds serum albumin can be formatted as a larger antigen-binding fragment of an antibody (e.g., formatted as a Fab, Fab′, F(ab)₂, F(ab′)₂, IgG, scFv).

In other embodiments of the invention described throughout this disclosure, instead of the use of a “dAb” in a fusion of the invention, it is contemplated that the skilled addressee can use a domain that comprises the CDRs of a dAb that binds specifically to serum albumin, e.g. CDRs of Dom7h-14, or Dom 7h-14-10 or Dom 7h-14-10 R108C, that binds serum albumin (e.g., the CDRs can be grafted onto a suitable protein scaffold or skeleton, eg an affibody, an SpA scaffold, an LDL receptor class A domain or an EGF domain). The disclosure as a whole is to be construed accordingly to provide disclosure of such domains in place of a dAb.

In certain embodiments, the invention provides a composition according to the invention that comprises a dual-specific ligand or multi-specific ligand that comprises a first dAb according to the invention that binds serum albumin e.g. any of those described herein e.g. Dom7h-14, and a second dAb that has the same or a different binding specificity from the first dAb and optionally in the case of multi-specific ligands further dAbs. The second dAb (or further dAbs) may optionally bind a different target e.g. FgFr 1c, or CD5 target.

In other embodiments of the invention, the dAb component can be any of the dAbs disclosed in WO 2008096158 or WO05118642 the details of which are incorporated by reference herein.

Thus, in one aspect, the invention provides the compositions of the invention for delivery by parenteral administration e.g. by subcutaneous, intramuscular or intravenous injection, inhalation, nasal delivery, transmucosal (e.g. sub-lingual) delivery, transcutaneous, transdermal, oral delivery, delivery to the GI tract of a patient, rectal delivery or ocular delivery. In one aspect, the invention provides the use of the fusions or conjugates of the invention in the manufacture of a medicament for delivery by subcutaneous injection or intramuscular, transdermal delivery, inhalation, intravenous delivery, nasal delivery, transmucossal delivery, oral delivery, delivery to the GI tract of a patient, rectal delivery or ocular delivery.

In one aspect, the invention provides a method for delivery to a patient by subcutaneous, intramuscular or intravenous injection, inhalation, nasal delivery, transmucosal (e.g. sub-lingual) delivery, transcutaneous, transdermal, oral delivery, delivery to the GI tract of a patient, rectal delivery or ocular delivery, wherein the method comprises administering to the patient a pharmaceutically effective amount of a fusion or conjugate of the invention.

In one aspect, the invention provides an oral, injectable, inhalable, nebulisable, topical or ocular formulation comprising a fusion or conjugate of the invention. The formulation can be a tablet, pill, capsule, liquid or syrup or ointment. In one aspect the compositions can be administered orally e.g. as a drink, for example marketed as a weight loss drink for obesity treatment. In one aspect, the invention provides a formulation for rectal delivery to a patient, the formulation can be provided e.g. as a suppository.

A composition for parenteral administration of GLP-1 compounds may, for example, be prepared as described in WO 03/002136 (incorporated herein by reference).

A composition for nasal administration of certain peptides may, for example, be prepared as generally described in European Patent No. 272097 (to Novo Nordisk A/S) or in WO 93/18785 (all incorporated herein by reference).

The term “subject” or “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species.

The invention also provides a kit for use in administering compositions according to the invention to a subject (e.g., human patient), comprising a composition of the invention, a drug delivery device and, optionally, instructions for use. The composition can be provided as a formulation, such as a freeze dried formulation. In certain embodiments, the drug delivery device is selected from the group consisting of a syringe, a pen injection device, an inhaler, an intranasal or ocular administration device (e.g., a mister, eye or nose dropper), and a needleless injection device.

The compositions (e.g conjugates or fusions) of this invention can be lyophilized for storage and reconstituted in a suitable carrier prior to use. Any suitable lyophilization method (e.g., spray drying, cake drying) and/or reconstitution techniques can be employed. It will be appreciated by those skilled in the art that lyophilisation and reconstitution can lead to varying degrees of antibody activity loss and that use levels may have to be adjusted to compensate. In a particular embodiment, the invention provides a composition comprising a lyophilized (freeze dried) composition as described herein. Preferably, the lyophilized (freeze dried) composition loses no more than about 20%, or no more than about 25%, or no more than about 30%, or no more than about 35%, or no more than about 40%, or no more than about 45%, or no more than about 50% of its activity (e.g., binding activity for serum albumin) when rehydrated. Activity is the amount of composition required to produce the effect of the composition before it was lyophilized. For example, the amount of conjugate or fusion needed to achieve and maintain a desired serum concentration for a desired period of time. The activity of the composition can be determined using any suitable method before lyophilization, and the activity can be determined using the same method after rehydration to determine amount of lost activity.

The invention also provides sustained release formulations comprising the compositions of the invention, such sustained release formulations can comprise the composition of the invention in combination with, e.g. hyaluronic acid, microspheres or liposomes and other pharmaceutically or pharmacalogically acceptable carriers, excipients and/or diluents. Such sustained release formulations can in the form of for example suppositories.

In one aspect, the invention provides a pharmaceutical composition comprising a composition of the invention, and a pharmaceutically or physiologically acceptable carrier, excipient or diluent.

### BRIEF DESCRIPTION OF THE DRAWINGS

FIG. 1: is an illustration of the amino acid sequences of (a) DAT0114 (SEQ ID NO 1), (b) DAT0115 (SEQ ID NO 2), (c) DAT0116 (SEQ ID NO 3), (d) DAT0117 (SEQ ID NO 4), (e) DAT0118 (SEQ ID NO 5), (f) DAT0119 (SEQ ID NO 6) (g) DAT0120 (SEQ ID NO 7) (h) Dom7h-14 (SEQ ID NO 8) ((Albudab™) (the CDRs are underlined), (i) GLP-1 7-37 A(8)G (SEQ ID NO 9), (j) exendin-4 (SEQ ID NO 10), (k) Helical linker (SEQ ID NO 11) (l) Gly-ser linker (SEQ ID NO 12), (m) Exendin 4, (G4S)3, linker DOM7h-14-10 fusion (DMS7139: SEQ ID NO 13), (n) Exendin 4, (G4S)3, linker DOM7h-11-15 fusion (DMS7143: SEQ ID NO 14), (o) DOM7h-14-10 (SEQ ID NO 15), (p) DOM7h-11-15 (Albudab™) (SEQ ID NO 16), (q) OmpT AWA signal peptide (leader) (SEQ ID NO 17), (r) DOM 7H-14-10 R108C mutant (Albudab™) (SEQ ID NO 18), (s) PYY 3-36 (with a lysine at position 10 derivatised with PEG) (SEQ ID NO 19) (t) 7h-11-15R108C (Albudab™) (SEQ ID NO 47); (u) DAT0116R108C:190 PYY (SEQ ID NO 48); (V) Genetic fusion of PYY-Dom 7h-14-10 albudab (SEQ ID NO 49)

FIG. 2: is an illustration of the nucleic acid sequences of: (a) DAT0114 (mammalian construct) (SEQ ID NO 20), (b) DAT0115 (mammalian construct) (SEQ ID NO 21), (c) DAT0115 (optimized for E. coli construct) (SEQ ID NO 22), (d) DAT0116 (mammalian construct) (SEQ ID NO 23), (e) DAT0116 (optimized for E. coli construct) (SEQ ID NO 24), (f) DAT0117 (mammalian construct) (SEQ ID NO 25), (g) DAT0117 (optimized for E. coli construct) (SEQ ID NO 26), (h) DAT0118 (mammalian construct) (SEQ ID NO 27), (i) DAT0119 (mammalian construct) (SEQ ID NO 28), (j) DAT0120 (mammalian construct) (SEQ ID NO 29), (k) Dom7h-14 (SEQ ID NO 30), (l) Exendin 4, (G4S)3, linker DOM7h-14-10 fusion (DMS7139: SEQ ID NO 31), (m) Exendin 4, (G4S)3, linker DOM7h-11-15 fusion (DMS7143: SEQ ID NO 32) (n) Dom 7h-14-10 (SEQ ID NO 33), (o) Dom 7h-11-15 (SEQ ID NO 34), (p) Omp AWA signal peptide (SEQ ID NO 35), (q) Dom 7h-14-10 R (108)C (SEQ ID NO 36).

FIG. 3: shows a peptide conjugate which is:
a Dom7h-14-10 (R108C) albudab conjugated to PYY3-36 via a lysine and 4 repeat PEG linker). This molecule was used in experiments detailed in examples 7-9.
(SEQ ID NO 37)

FIG. 4: shows change in body weight over time in DIO mice treated with peptide-AlbudAbs.

FIG. 5: shows change in food intake over time in DIO mice treated with peptide-AlbudAbs.

FIG. 6 shows body fat % in DIO mice treated with peptide-AlbudAbs. (baseline and at day 15).

FIG. 7: shows change in body fat and lean mass in DIO mice (baseline vs 15 days) in mice treated with peptide-AlbudAbs.

FIG. 8: shows measurements of endocrine analytes in DIO mice treated with peptide-AlbudAbs.

FIG. 9: shows changes in histopathology in the liver on DIO mice treated with combinations of peptide-AlbudAbs and controls.

FIG. 10: shows measurements of glycosylated Haemoglobin Alc in db/db mice treated with peptide-AlbudAbs.

FIG. 11: shows the change in % HbAlc (baseline vs day 16) in db/db mice treated with peptide-AlbudAbs.

FIG. 12: shows plasma insulin levels (at day 16) in db/db mice treated with peptide-AlbudAbs.

FIG. 13: shows change in body weight over time in db/db mice treated with peptide-AlbudAbs.

FIG. 14: shows change in food intake over time in db/db mice treated with peptide-AlbudAbs.

FIG. 15: shows the amino acid sequences of leaders: (a) ompA (E. coli derived) (SEQ ID NO 38), (b) ompA-AMA (artificial sequence) (SEQ ID NO 39), (c) ompA-AWA (artificial sequence) (SEQ ID NO 40), (d) ompT (E. coli derived) (SEQ ID NO 41), (e) ompT-AMA (artificial sequence) (SEQ ID NO 42), (f) GAS (S. cerevisiae derived) (SEQ ID NO 43), (g) GAS-AMA (artificial sequence) (SEQ ID NO 44), (h) GAS-AWA (artificial sequence) (SEQ ID NO 45) (i) Pel B ((Erwinia carotovora) (SEQ ID NO 46).

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The term “insulinotropic agent” as used herein means a compound which is able to stimulate, or cause the stimulation of, the synthesis or expression of, or the activity of the hormone insulin. Known examples of insulinotropic agents include but are not limited to e.g. glucose, GIP, GLP, Exendin (e.g. exendin-4 and exendin-3), PYY (e.g. 3-36 PYY) and OXM.

The term “incretin” as used herein means a type of gastrointestinal hormone that causes an increase in the amount of insulin released when glucose levels are normal or particularly when they are elevated. By way of example they include GLP-1, GIP, OXM, VIP, and PP (pancreatic polypeptide).

Gut peptides are a class of peptides released from various cells in different parts of the gut that provide a signaling function, PYY is also an example of a gut peptide.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide or they can be within the peptide. A simple system is used to describe analogues of GLP-1: For example GLP-1 A8G (7-37 amino acids) designates a GLP-1 analogue wherein the naturally occurring alanine at position 8 has been substituted with a glycine residue. Formulae of peptide analogs and derivatives thereof are drawn using standard single letter abbreviation for amino acids used according to IUPAC-IUB nomenclature.

As used herein “fragment,” when used in reference to a polypeptide, is a polypeptide having an amino acid sequence that is the same as part but not all of the amino acid sequence of the entire naturally occurring polypeptide. Fragments may be “free-standing” or comprised within a larger polypeptide of which they form a part or region as a single continuous region in a single larger polypeptide. By way of example, a fragment of naturally occurring GLP-1 would include amino acids 7 to 36 of naturally occurring amino acids 1 to 36. Furthermore, fragments of a polypeptide may also be variants of the naturally occurring partial sequence. For instance, a fragment of GLP-1 comprising amino acids 7-30 of naturally occurring GLP-1 may also be a variant having amino acid substitutions within its partial sequence.

Examples of suitable insulinotropic agents of the invention include GLP-1, GLP-1 derivatives, GLP-1 analogues, or a derivative of a GLP-1 analogue. In addition they include Exendin-4, Exendin-4 analogues and Exendin-4 derivatives or fragments and Exendin-3, Exendin-3 derivatives and Exendin-3 analogues, PYY PYY-1 derivatives, PYY-1 analogues, or a derivative of a PYY-1 analogue, PYY fragments (e.g. 3-36 and/or 13-36 PYY).

The term “GLP-1 ” as used herein means GLP-1 (7-37), GLP-1 (7-36), GLP-1 (7-35), GLP-1 (7-38), GLP-1 (7-39), GLP-1 (7-40), GLP-1 (7-41), a GLP-1 analogue, a GLP-1 peptide , a GLP-1 derivative or mutant or fragment or a derivative of a GLP-1 analogue. Such peptides, mutants, analogues and derivatives are insulinotropic agents.

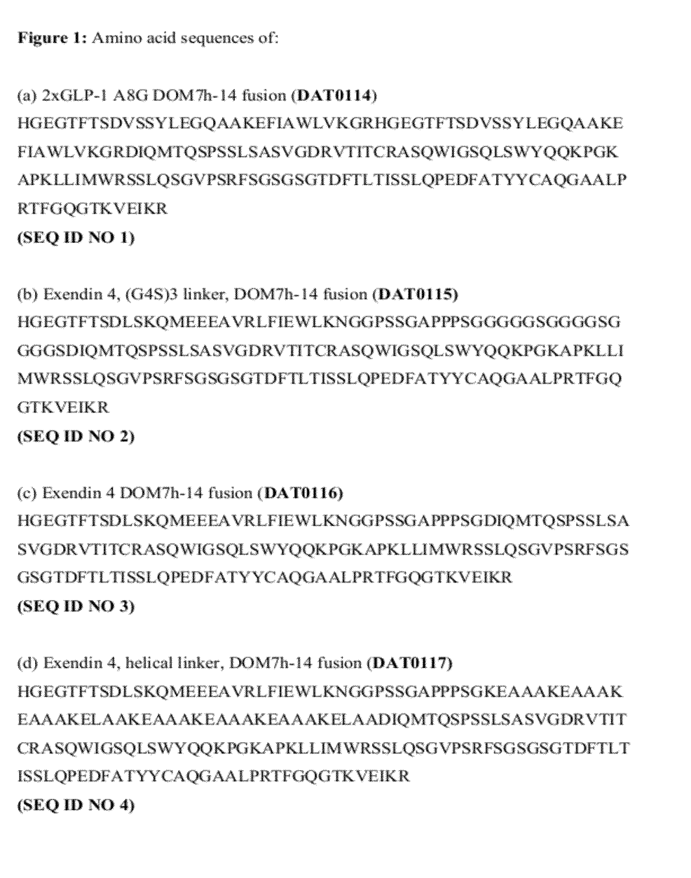

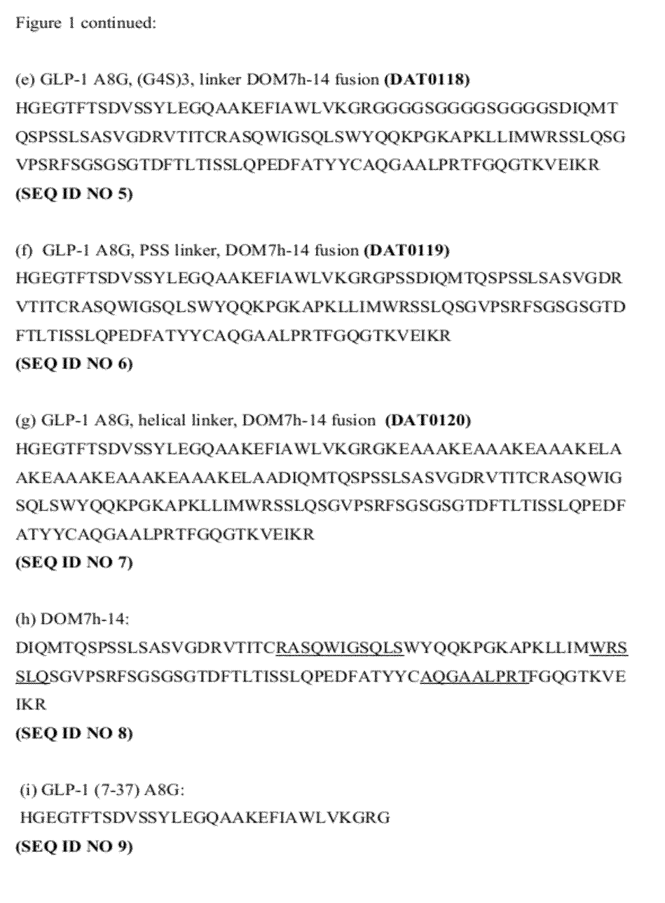

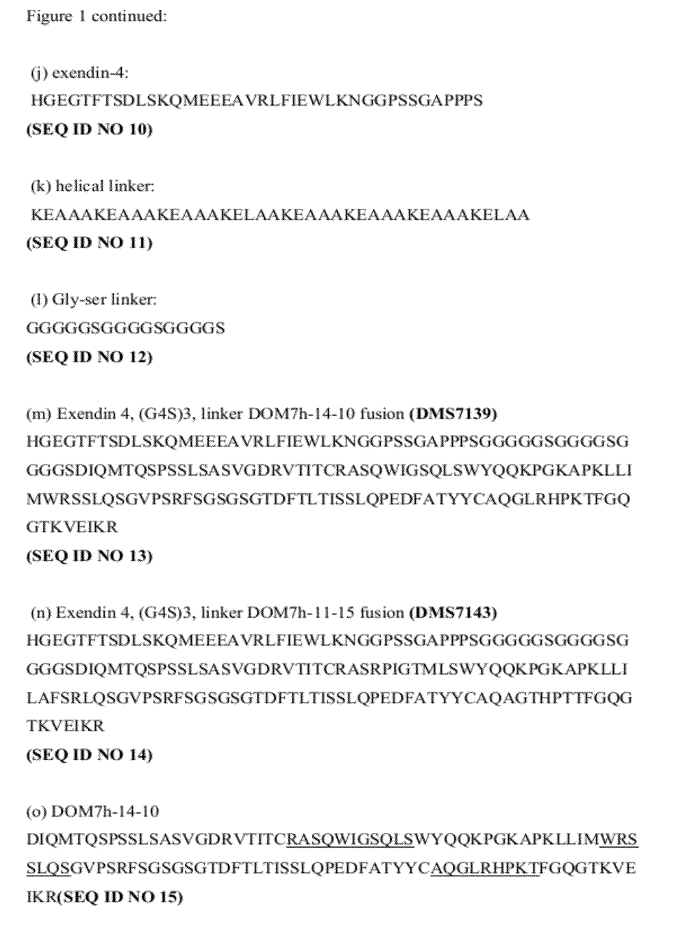

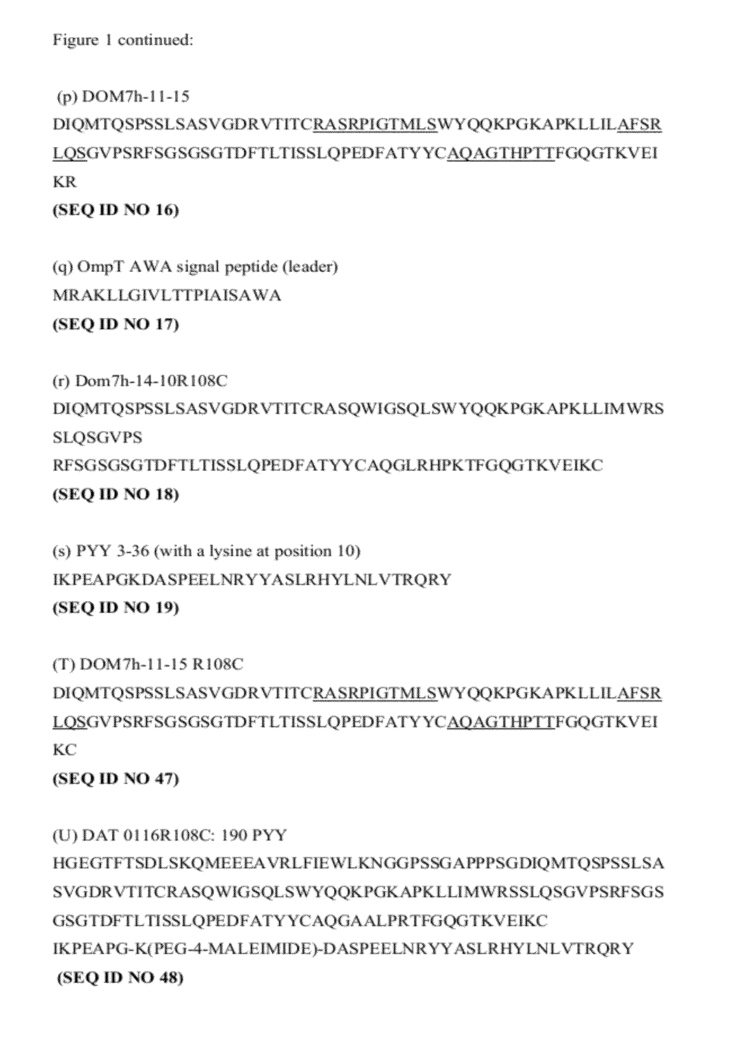

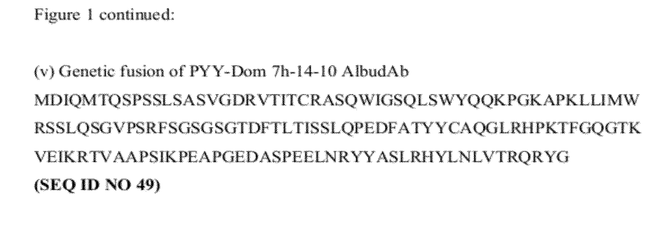

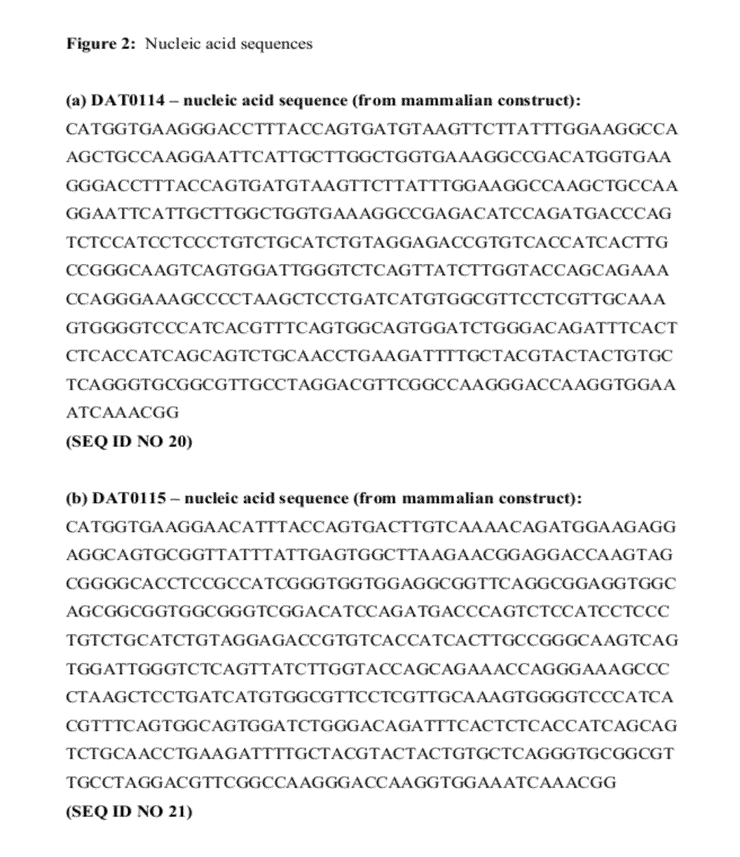

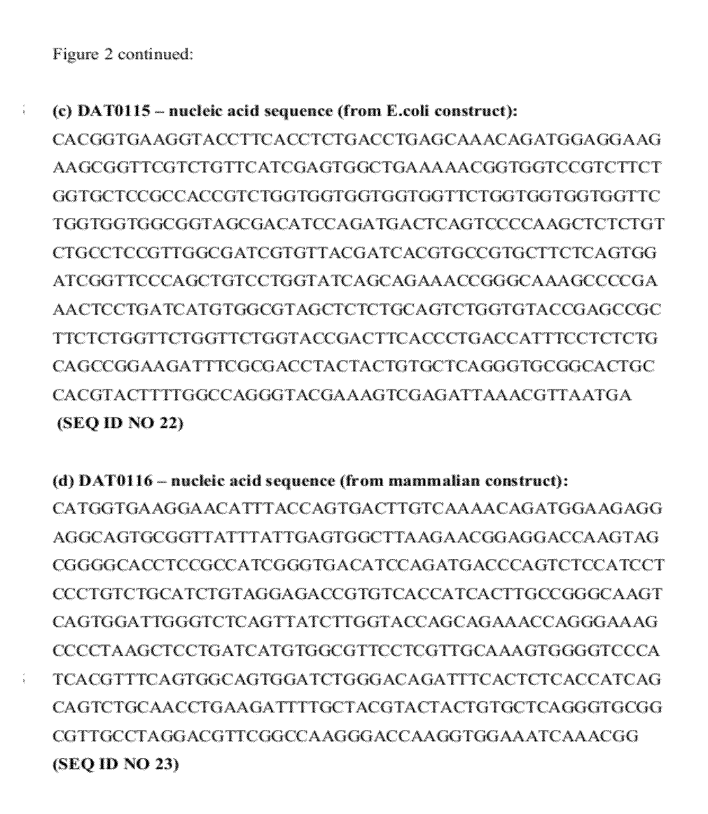

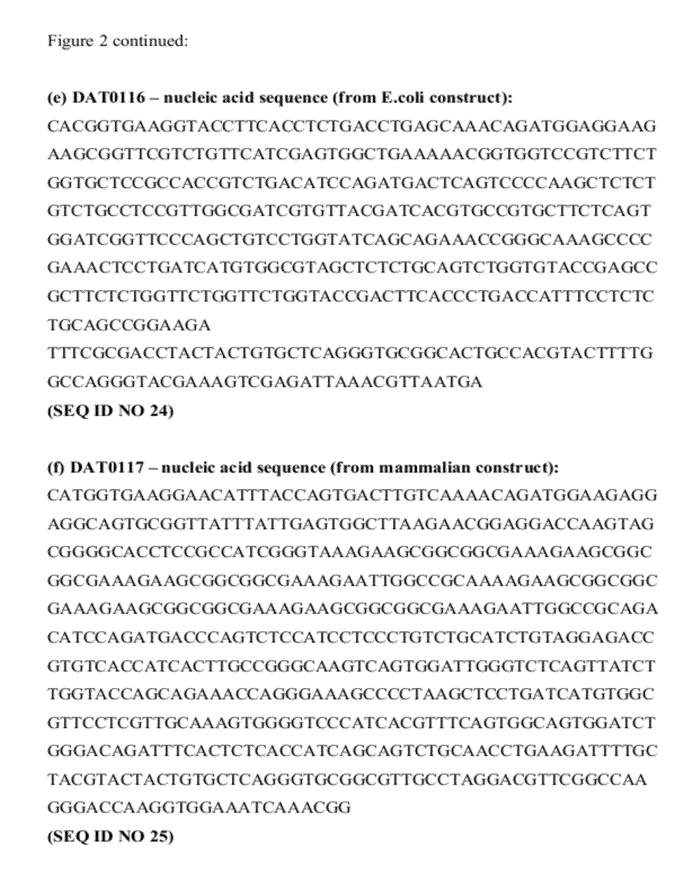

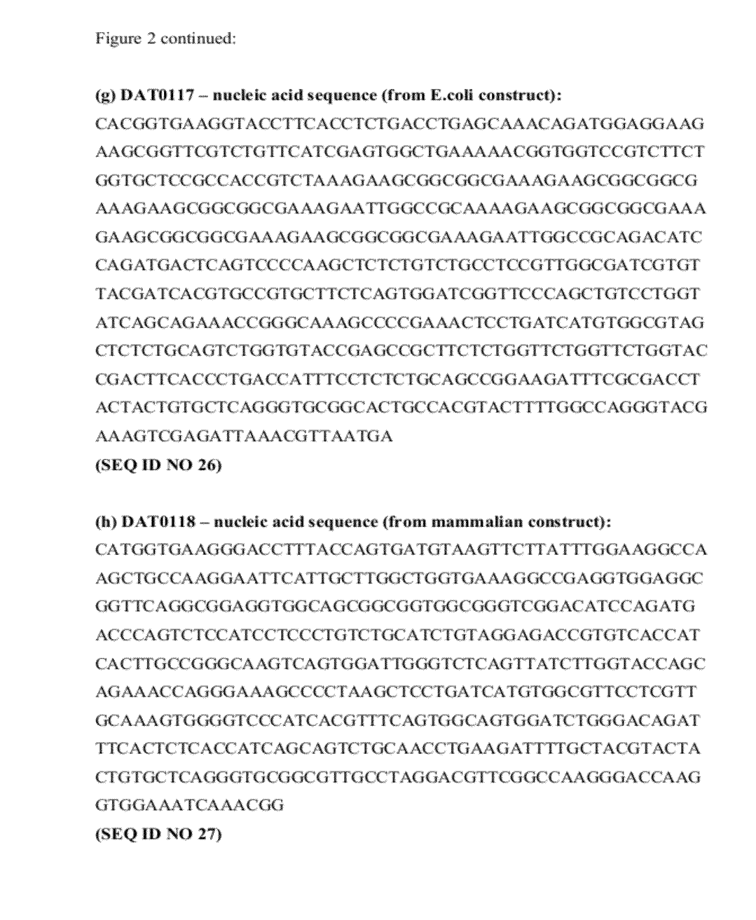

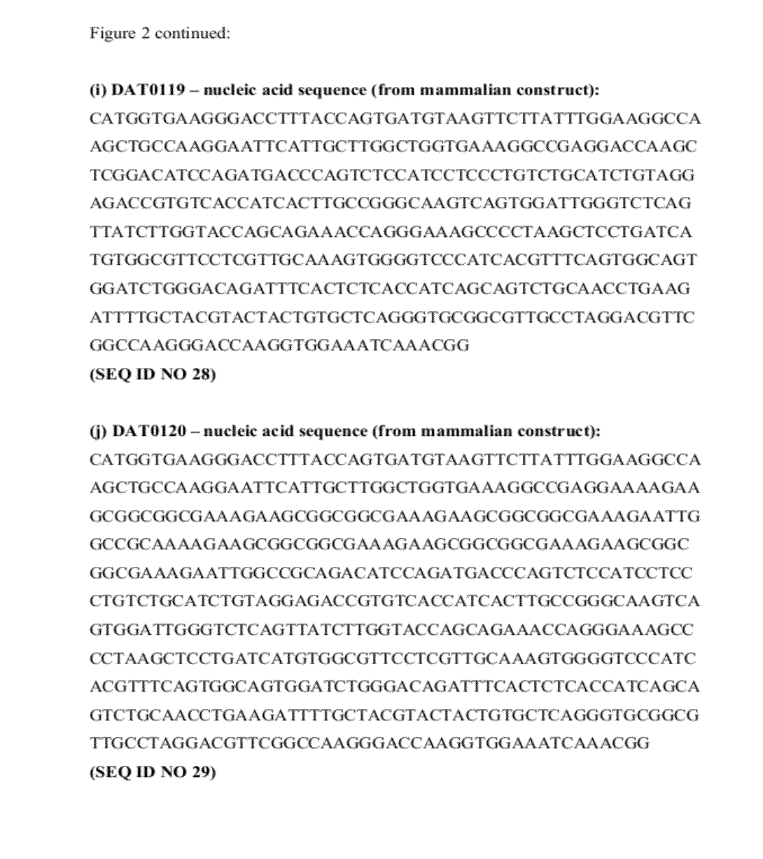

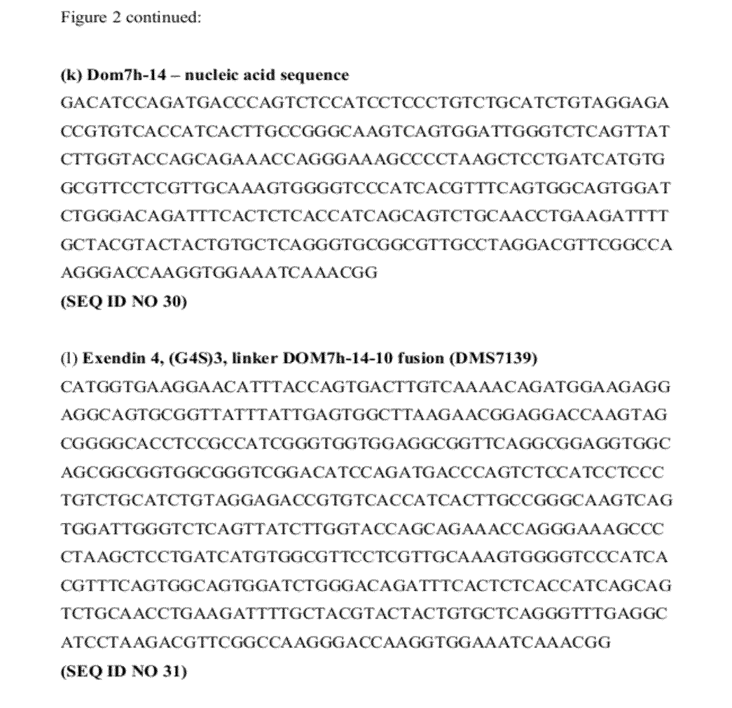

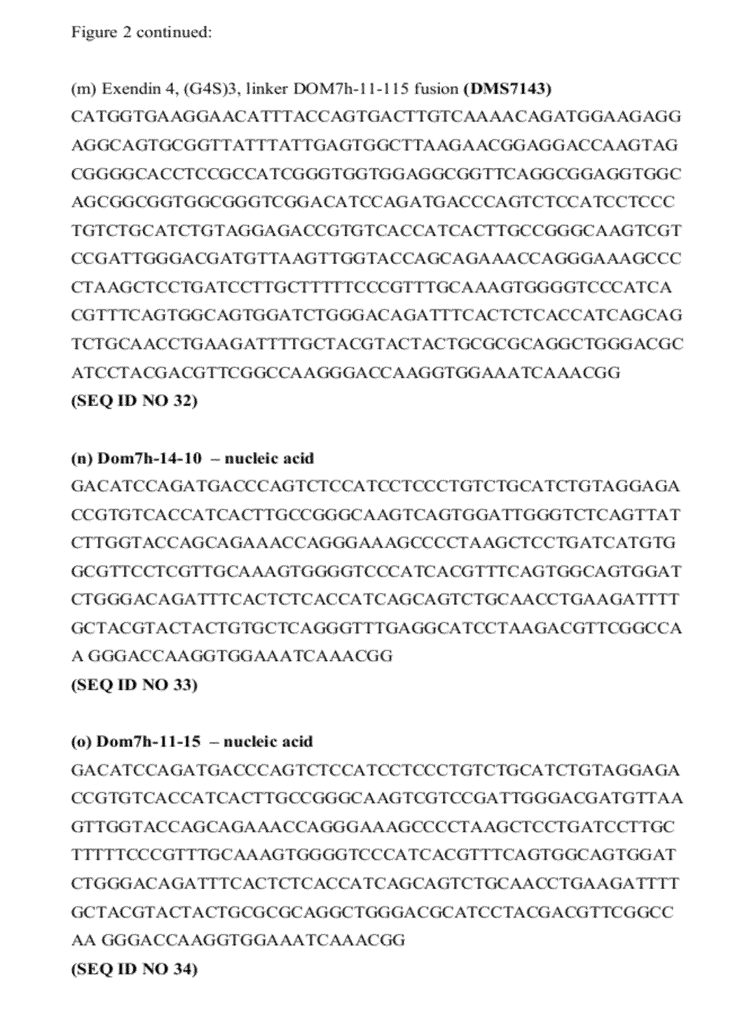

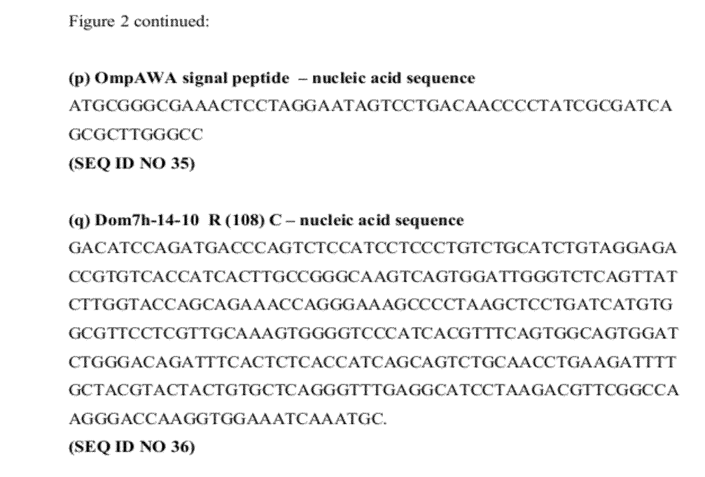

For example the GLP-1 can be GLP-1 (7-37) A8G mutant with the amino acid sequence shown in FIG. 1 (i): SEQ ID NO 9.

Further GLP-1 analogues are described in International Patent Application No. 90/11296 (The General Hospital Corporation) which relates to peptide fragments which comprise GLP-1 (7-36) and functional derivatives thereof and have an insulinotropic activity which exceeds the insulinotropic activity of GLP-1 (1-36) or GLP-1 (1-37) and to their use as insulinotropic agents (incorporated herein by reference, particularly by way of examples of drugs for use in the present invention).

International Patent Application No. WO 91/11457 (Buckley et al.) discloses analogues of the active GLP-1 peptides 7-34,7-35, 7-36, and 7-37 which can also be useful as GLP-1 drugs according to the present invention (incorporated herein by reference, particularly by way of examples of drugs or agents for use in the present invention).

The term “exendin-4 peptide” as used herein means exendin-4 (1-39), an exendin-4 analogue, a fragment of exendin-4 peptide, an exendin-4 derivative or a derivative of an exendin-4 analogue. Such peptides, fragments, analogues and derivatives are insulinotropic agents. The amino acid sequence of exendin-4 (1-39) is shown in FIG. 1 (j): SEQ ID NO 10.

Further Exendin-analogs that are useful for the present invention are described in PCT patent publications WO 99/25728 (Beeley et al.), WO 99/25727 Beeley et al.), WO 98/05351 (Young et al.), WO 99/40788 (Young et al.), WO 99/07404 (Beeley et al), and WO 99/43708 (Knudsen et al) (all incorporated herein by reference, particularly by way of examples of drugs for use in the present invention).

The term PYY as used herein refers to the Peptide YY which is a short (36 amino acid) protein released in response to feeding. PYY concentration in the circulation increases postprandially and decreases on fasting. Fragments (e.g. active fragments) of the PYY peptide are also useful for the present invention e.g. 3-36, 13-36 as are PYY analogues and derivatives which retain activity.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, “display system” refers to a system in which a collection of polypeptides or peptides are accessible for selection based upon a desired characteristic, such as a physical, chemical or functional characteristic. The display system can be a suitable repertoire of polypeptides or peptides (e.g., in a solution, immobilized on a suitable support). The display system can also be a system that employs a cellular expression system (e.g., expression of a library of nucleic acids in, e.g., transformed, infected, transfected or transduced cells and display of the encoded polypeptides on the surface of the cells) or an acellular expression system (e.g., emulsion compartmentalization and display). Exemplary display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and a nucleic acid encoding the selected polypeptide or peptide can be readily isolated or recovered. A number of display systems that link the coding function of a nucleic acid and physical, chemical and/or functional characteristics of a polypeptide or peptide are known in the art, for example, bacteriophage display (phage display, for example phagemid display), ribosome display, emulsion compartmentalization and display, yeast display, puromycin display, bacterial display, display on plasmid, covalent display and the like. (See, e.g., EP 0436597 (Dyax), U.S. Pat. No. 6,172,197 (McCafferty et al.), U.S. Pat. No. 6,489,103 (Griffiths et al.).)

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “target ligand” refers to a ligand which is specifically or selectively bound by a polypeptide or peptide. For example, when a polypeptide is an antibody or antigen-binding fragment thereof, the target ligand can be any desired antigen or epitope. Binding to the target antigen is dependent upon the polypeptide or peptide being functional.

As used herein an antibody refers to IgG, IgM, IgA, IgD or IgE or a fragment (such as a Fab , F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of other V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one embodiment, each individual organism or cell contains only one or a limited number of library members. In one embodiment, the nucleic acids are incorporated into expression vectors, in order to allow expression of the polypeptides encoded by the nucleic acids. In an aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

As used herein, the term “dose” refers to the quantity of fusion or conjugate administered to a subject all at one time (unit dose), or in two or more administrations over a defined time interval. For example, dose can refer to the quantity of fusion or conjugate administered to a subject over the course of one day (24 hours) (daily dose), two days, one week, two weeks, three weeks or , one month, two months, three months, or six or more months (e.g., by a single administration, or by two or more administrations). The interval between doses can be any desired amount of time.

The phrase, “half-life,” refers to the time taken for the serum or plasma concentration of the fusion or conjugate to reduce by 50%, in vivo, for example due to degradation and/or clearance or sequestration by natural mechanisms. The compositions of the invention are stabilized in vivo and their half-life increased by binding to serum albumin molecules e.g. human serum albumin (HSA) which resist degradation and/or clearance or sequestration. These serum albumin molecules are naturally occurring proteins which themselves have a long half-life in vivo. The half-life of a molecule is increased if its functional activity persists, in vivo, for a longer period than a similar molecule which is not specific for the half-life increasing molecule. For example, a composition of the invention comprising a dAb specific for human serum albumin (HSA) and incretin and/or insulinotropic and/or gut peptide molecules such as GLP-1, PYY or exendin is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

As used herein, “hydrodynamic size” refers to the apparent size of a molecule (e.g., a protein molecule, ligand) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein.

Calculations of “homology” or “identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. Amino acid and nucleotide sequence alignments and homology, similarity or identity, as defined herein may be prepared and determined using the algorithm BLAST 2 Sequences, using default parameters (Tatusova, T. A. et al., FEMS Microbiol Lett, 174:187-188 (1999).

Post translational modifications of amino acid sequences: it is known that post translational modification of amino acid sequences can occur naturally these can comprise for example deamidation or N terminal cyclisation or addition or deletion of residues. The invention therefore includes variants of the sequences disclosed herein resulting from such post translational modifications e.g. deamidated forms of the sequences.

### Nucleic Acids, Host Cells:

The invention relates to isolated and/or recombinant nucleic acids encoding the compositions e.g. fusions, of the invention that are described herein.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid).

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR).

The invention also relates to a recombinant host cell e.g.mammalian or microbial, which comprises a (one or more) recombinant nucleic acid or expression construct comprising nucleic acid(s) encoding a composition e.g. fusion, of the invention as described herein. There is also provided a method of preparing a composition, e.g. fusion, of the invention as described herein, comprising maintaining a recombinant host cell e.g.mammalian or microbial, of the invention under conditions appropriate for expression of the fusion polypeptide. The method can further comprise the step of isolating or recovering the fusion, if desired.

For example, a nucleic acid molecule (i.e., one or more nucleic acid molecules) encoding a composition of the invention e.g. a fusion polypeptide of the invention, or an expression construct (i.e., one or more constructs) comprising such nucleic acid molecule(s), can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g., in the presence of an inducer, in a suitable animal, in suitable culture media supplemented with appropriate salts, growth factors, antibiotics, nutritional supplements, etc.), whereby the encoded peptide or polypeptide is produced. If desired, the encoded peptide or polypeptide can be isolated or recovered (e.g., from the mammal, the animal, the host cell, medium, milk). This process encompasses expression in a host cell of a transgenic animal (see, e.g., WO 92/03918, GenPharm International). The peptide or fusion protein or conjugate can subsequently be further modified e.g. chemically or enzymatically either in the expression host, in the culture medium, during or after purification e.g. via amidation of the C terminus.

The compositions, e.g. fusion polypeptides, of the invention described herein can also be produced in a suitable in vitro expression system, e.g. by chemical synthesis or by any other suitable method.

As described and exemplified herein, compositions e.g. fusions and conjugates of the invention, generally bind serum albumin with high affinity.

For example, the fusions or conjugates can bind human serum albumin with an affinity (KD; KD=K_{off}(kd)/Kₒₙ(ka) [as determined by surface plasmon resonance) of about 5 micromolar to about 100 pM , e.g. about 1 micromolar to about 100 pM e.g. 400-800 nm e.g. about 600 nm.

The compositions e.g. fusions or conjugates, of the invention can be expressed in E. coli or in Pichia species (e.g., P. pastoris). In one embodiment, the fusion is secreted in a quantity of at least about 0.5 mg/L when expressed in E. coli or in Pichia species (e.g., P. pastoris); or in mammalian cell culture (e.g. CHO, or HEK 293 cells). Although, the fusions or conjugates described herein can be secretable when expressed in E. coli or in Pichia species or mammalian cells they can be produced using any suitable method, such as synthetic chemical methods or biological production methods that do not employ E. coli or Pichia species.

In certain embodiments, compositions of the invention are efficacious in animal models of such as those described in WO 2006 /059106 (e.g. at pages 104-105 of published WO 2006 /059106) or those described in the examples herein, when an effective amount is administered. Generally an effective amount is about 0.0001 mg/kg to about 10 mg/kg (e.g., about 0.001 mg/kg to about 10 mg/kg, e.g. about 0.001 mg/kg to about 1 mg/kg, e.g. about 0.01 mg/kg to about 1 mg/kg, e.g. about 0.01 mg/kg to about 0.1 mg/kg). The models of disease are recognized by those skilled in the art as being predictive of therapeutic efficacy in humans.

Generally, the present compositions of the invention will be utilised in purified form together with pharmacologically or physiologically appropriate carriers. Typically, these carriers can include aqueous or alcoholic/aqueous solutions, emulsions or suspensions, any including saline and/or buffered media. Parenteral vehicles can include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates, sucrose, trehalose, sorbitol, detergents such as tween-20 or tween-80.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The route of administration of pharmaceutical compositions according to the invention may be any of those commonly known to those of ordinary skill in the art. For therapy, the drug fusions or conjugates of the invention can be administered to any patient in accordance with standard techniques.

The administration can be by any appropriate mode, including parenterally, intravenously, transmucosal delivery (e.g. sub-lingual), by subcutaneous injection, intramuscularly, intraperitoneally, orally, transdermally, transmucosally, via the pulmonary route, via nasal delivery, GI delivery, rectal delivery, or ocular delivery or also, appropriately, by direct infusion with a catheter. The dosage and frequency of administration will depend on the age, sex and condition of the patient, concurrent administration of other drugs, counterindications and other parameters to be taken into account by the clinician. Administration can be local or systemic as indicated.

The compositions of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

For prophylactic applications, e.g. when administering to individuals with pre-diabetes or with insulin resistance, compositions containing the present fusions or conjugates may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a composition of the invention is administered to treat, suppress or prevent disease, it can be administered up to four times per day, once per day, twice weekly, once weekly, once every two weeks, once a month, or once every two months, once every three months, once every six months, or at a longer interval, at a dose of, for example about 0.0001 mg/kg to about 10 mg/kg (e.g., about 0.001 mg/kg to about 10 mg/kg e.g. about 0.001 mg/kg to about 1 mg/kg e.g. about 0.01 mg/kg to about 1 mg/kg, e.g. about 0.01 mg/kg to about 0.1 mg/kg).

Treatment or therapy performed using the compositions described herein is considered “effective” if one or more symptoms or signs are reduced or alleviated (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the precise nature of the disease or disorder targeted, but can be measured by an ordinarily skilled clinician or technician.

Similarly, prophylaxis performed using a composition as described herein is “effective” if the onset or severity of one or more symptoms or signs is delayed, reduced or abolished relative to such symptoms in a similar individual (human or animal model) not treated with the composition.

The compositions of the present invention may be administered in conjunction with other therapeutic or active agents e.g. other polypeptides or peptides or small molecules. These further agents can include various drugs, such as for example metformin, insulin, glitazones (e.g. rosaglitazone), immunosuppresives, immunostimulants.

The compositions of the invention can be administered and/or formulated together with one or more additional therapeutic or active agents. When a composition of the invention is administered with an additional therapeutic agent, the fusion or conjugate can be administered before, simultaneously, with, or subsequent to administration of the additional agent. Generally, the composition of the invention and the additional agent are administered in a manner that provides an overlap of therapeutic effect.

### Half Life:

Increased half-life of the insulinotropic and/or incretin and/or gut peptide molecule e.g. the GLP-1, PYY or exendin ligand is useful in in vivo applications. The invention solves this problem by providing increased half-life of the insulinotropic agent and/or incretin and/or gut peptide drug e.g. GLP and exendin, in vivo and consequently longer persistence times in the body of the functional activity of these molecules.

As described herein, compositions of the invention can have dramatically prolonged in vivo serum or plasma half-life and/or increased AUC and/or increased mean residence time (MRT), as compared to insulinotropic and/or incretin and/or gut peptide molecule alone. In addition, the activity of the insulinotropic and/or incretin and/or gut peptide molecule is generally not substantially altered in the composition of the invention (e.g., the conjugate, or the fusion). However, some change in the activity of compositions of the invention compared to insulinotropic and/or incretin and/or gut peptide molecule alone is acceptable and is generally compensated for by the improved pharmacokinetic properties of the compositions of the invention. For example, compositions of the invention may bind the target with lower affinity than incretin/insulinotropic agent alone, but have about equivalent or superior efficacy in comparison to incretin/insulinotropic agent alone due to the improved pharmacokinetic properties (e.g., prolonged in vivo serum half-life, larger AUC) of the composition. In addition, due to the increased half life of the compositions of the invention they can be administed less frequently than the insulinotropic agent and/or incretin and/or gut peptide drug alone e.g. they can be given to patients once a month or once a week, and they also attain a more constant level of insulinotropic and/or incretin and/or gut peptide agent in the blood than administration of insulinotropic and/or incretin and/or gut peptide alone, so achievin_{g} the desired therapeutic or prophylactic effect.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Half lives (t1/2 alpha and t1/2 beta) and AUC and MRT can be determined from a curve of plasma or serum concentration of ligand against time. The WinNonlin analysis package (available from Pharsight Corp., Mountain View, Calif. 94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. In addition a non-compartmental fitting model that is well known in the art can also be used to determine half life.

In one embodiment, the present invention provides a composition, comprising fusion(s) or conjugate(s), according to the invention wherein the fusion or conjugate has an elimination halflife e.g. in human subjects, in the range of about 12 hours or more, e.g.' about 12 hours to about 21 days, e.g. about 24 hours to about 21 days, e.g. about 2-8 days e.g. about 3-4 days.

Compositions of the invention, i.e. those comprising the fusions and conjugates described herein, provide several further advantages. The Domain antibody component is very stable, is small relative to antibodies and other antigen-binding fragments of antibodies, can be produced in high yields by expression in E. coli or yeast (e.g., Pichia pastoris), or mammalian cells (e.g. CHO cells) and antigen-binding fragments of antibodies that bind serum albumin can be easily selected from libraries of human origin or from any desired species. Accordingly, compositions of the invention that comprise the dAb that binds serum albumin can be produced more easily than therapeutics that are generally produced in mammalian cells (e.g., human, humanized or chimeric antibodies) and dAbs that are not immunogenic can be used (e.g., a human dAb can be used for treating or diagnosing disease in humans).

The immunogenicity of the insulinotropic and/or incretin and/or gut peptide molecule(s) can be reduced when it is part of a drug composition that contains a dAb that binds serum albumin. Accordingly, the invention provides a compositions which can be less immunogenic (than e.g. the insulinotropic and/or incretin and/or gut peptide molecules alone) or which can be substantially non-immunogenic in the context of a drug composition that contains a dAb that binds serum albumin. Thus, such compositions can be administered to a subject repeatedly over time with minimal loss of efficacy due to the elaboration of anti-drug antibodies by the subject's immune system.

Additionally, the compositions described herein can have an enhanced safety profile and fewer side effects than the insulinotropic and/or incretin and/or gut peptide agents alone. For example, as a result of the serum albumin-binding activity of the dAb, the fusions and conjugates of the invention have enhanced residence time in the vascular circulation. Additionally, the compositions of the invention are substantially unable to cross the blood brain barrier and to accumulate in the central nervous system following systemic administration (e.g., intravascular administration). Accordingly, the compositions of the invention can be administered with greater safety and reduced side effects in comparison to the insulinotropic and/or incretin and/or gut peptide agent alone alone. Similarly, the compositions of the invention can have reduced toxicity toward particular organs (e.g., kidney or liver) than drug alone.

### EXAMPLES

### Example 1

### Expression of Genetic Fusions of GLP-1 (A8G) or Exendin-4 and DOM7h-14 AlbudAb

Either exendin-4 or GLP-1 (7-37), with alanine at position 8 replaced by glycine ([Gly⁸] GLP-1), was cloned as a fusion with DOM7h-14 (a domain antibody (dAb) which binds serum albumin (albudab) with an amino acid sequence shown below) into the pTT-5 vector (obtainable from CNRC, Canada). In each case the GLP-1 or exendin-4 was at the 5′ end of the construct and the dAb at the 3′ end. In total, 7 constructs (DAT0114, DAT 0115, DAT0116, DAT 0117, DAT 0118, DAT 0119, DAT 0120) were made with the amino acid sequences shown in FIG. 1 (A-G). Between GLP-1 or exendin 4 and the dAb there was either no linker, a gly-ser linker (G4S×3), or a helical linker. “Design of the linkers which effectively separate domains of a bifunctional fusion protein.” Protein Eng 14(8): 529-32.456) or a linker composed of a second GLP-1 moiety between the GLP-1 or exendin 4 and the dAb. The linkers were included as spacers to separate the GLP-1 or exendin 4 spatially from the dAb to prevent steric hindrance of the binding between the GLP-1 or exendin-4 and the GLP-1 receptor. The sequences of the constructs are shown in FIG. 1 (A-G) SEQ ID NOS 1-7.

Endotoxin free DNA was prepared in E. coli using alkaline lysis (using the endotoxin free plasmid Giga kit, obtainable from Qiagen Calif.) and used to transfect HEK293E cells (obtainable from CNRC, Canada). Transfection was into 250 ml/flask of HEK293E cells at 1.75×10⁶ cells/ml using 333 ul of 293 fectin (Invitrogen) and 250 ug of DNA per flask and expression was at 30° C. for 5 days. The supernatant was harvested by centrifugation and purification was by affinity purification on protein L. Protein was batch bound to the resin, packed on a column and washed with 10 column volumes of PBS. Protein was eluted with 50 ml of 0.1 M glycine pH2 and neatralised with Tris pH8. Protein of the expected size was identified on an SDS-PAGE gel. Sizes are shown in the table 1 below

TABLE 1
Molecular weights of DAT0114, DAT 0115, DAT0116,
DAT 0117, DAT 0118, DAT 0119, DAT 0120 constructs

| Fusion protein | Expected MW |
|---|---|
| DAT0114 | 18256 |
| DAT0115 | 16896 |
| DAT0116 | 15950 |
| DAT0117 | 19798 |
| DAT0118 | 15936 |
| DAT0119 | 15318 |
| DAT0120 | 18895 |

### Example 2

### Showing that GLP-1 and Exendin-4 AlbudAb Fusions Bind Serum Albumin

GLP-1 and Exendin-4 AlbudAb fusions were analysed by surface plasmon resonance (Biacore AB obtainable from GE Healthcare) to obtain information on affinity. The analysis was performed using a CM5 Biacore chip (carboxymethylated dextran matrix) that was coated with serum albumin. About 1000 resonance units (RUs) of each serum albumin to be tested (human, rat and mouse serum albumin) was immobilised in acetate buffer pH 5.5. Flow cell 1 of the Biocore AB was an uncoated, blocked negative control, flow cell 2 was coated with Human serum albumin (HSA) (815 RUs) flow cell 3 was coated with Rat serum albumin (RSA)(826RUs) and flow cell 4 was coated with Mouse serum albumin (MSA) (938 RUs). Each fusion molecule tested was expressed in mammalian tissue culture as described in the example above.

A range of concentrations of the fusion molecule were prepared (in the range 16 nM to 2 μM) by dilution into BIACORE HBS-EP buffer (0.01 M HEPES, pH7.4, 0.15 M NaCl, 3 mM EDTA, 0.005% surfactant P20) and flowed across the BIACORE chip.

Affinity (KD) was calculated from the BIACORE traces by fitting on-rate and off-rate curves to traces generated by concentrations of dAb in the region of the KD. Affinities (KD) are summarised in the following table 2:

TABLE 2
Binding of GLP-1 and exendin-4 AlbudAb
to human, rat and mouse serum albumins

| | DAT 0120: GLP-1 | |
|---|---|---|
| | (7-37) A8G, | DAT 0117: 2xGLP-1 |
| | helical linker, | (7-37) A8G |
| | DOM7h-14 fusion | DOM7h-14 fusion |
| HSA | 110 nM | 150 nM |
| RSA | 800 nM | 700 nM |
| MSA | 110 nM | 130 nM |

The results above demonstrate that the fusion molecules retain the ability to bind to all types of serum albumin and this indicates that they are likely to have an extended half life in vivo.

### Example 3

### GLP-1 and exendin-4 AlbudAb Fusions are Active in a GLP-1 Receptor Binding Assay (GLP-1R BA):

Fusions were buffer exchanged into 100 mM NaV1, 20 mM citrate pH 6.2. Meanwhile,CHO 6CRE GLP1R cells (CHO K1 cells (obtainable from the American Type Tissue Collection, ATCC) stably transfected with 6 cAMP response element driving a luciferase reporter gene and also with the human GLP-1 receptor) were seeded at 2×10⁵cells/mL in suspension media. Suspension culture was maintained for 24 hours. Cells were then diluted into 15 mM HEPES buffer (obtainable from Sigma), containing 2 mM L glutamine (2.5×10⁵ cells/ml) and dispensed into 384-well plates containing 10 ul/well of the compound to be assayed. After the addition of assay control, plates were returned to the incubator for 3 h at 37° C. and 5% CO2. After the incubation, steady glo luciferase substrate (obtainable from Promega) was added to the wells as described in the kit and the plates sealed with self-adhesive plate seals (Weber Marking Systems Inc. Cat. No. 607780). Plates were placed in the reader (Viewlux, Perkin Elmer) and pre-incubated for 5 minutes prior to reading the fluorescence and plotting of results. Compound was assayed at a range of concentrations in the presence and absence of 10 uM albumin, allowing a dose response curve to be fitted with and without the albumin. EC50s were calculated and are summarised in the following table 3:

TABLE 3
Activity of GLP-1 and exendin-4 AlbudAb fusions
in a GLP-1 receptor binding assay (GLP-1R BA)

| | | GLP-1R BA |
|---|---|---|
| | GLP-1R BA | (10 uM albumin) |
| | EC₅₀ (pM) n = 3 | EC₅₀ (pM) n = 2 |
| DAT 0115: Exendin 4 | 8 | 38 |
| (G4S)3 DOM7h-14 fusion | | |
| DAT 0116: Exendin 4 | 12 | 72 |
| DOM7h-14 fusion | | |
| DAT 0117: Exendin 4, | 4 | 15 |
| helical linker, | | |
| DOM7h-14 fusion | | |
| DAT 0120: GLP-1 A8G, | 18 | 127 |
| helical linker, | | |
| DOM7h-14 fusion | | |
| GLP-1 7-36 | 16 | 18 |
| Exendin-4 | 1.0 | 0.82 |

The results above demonstrate that all of the fusion molecules tested retain potency for binding to the GLP-1 receptor. The results also demonstrate that this potency is retained in the presence of serum albumin. Hence, these fusion molecules are likely to retain the ability to bind the GLP-1 receptor in vivo.

### Example 4

### Expression of DAT0115, DAT0116, DAT0117 and DAT0120 in HEK 293 Mammalian Tissue Culture Followed by Purification by Protein L Affinity Capture and Ion Exchange Chromatography

The aim of this experiment was to produce protein for in vivo and in vitro characterisation. Protein was expressed in mammalian tissue culture in HEK 293E cells from the pTT-5 vector as described in the previously. Briefly, endotoxin free DNA was prepared and purified and used to transfect HEK293E cells. Protein expression was for 5 days at 30° C. in a shaking incubator and cultures were spun down and supernatant (containing the protein of interest) harvested. Protein was purified from the supernatant by affinity capture on protein L agarose streamline affinity resin (resin GE Healthcare, protein L coupled in house). Resin was then washed with approximately 10 column volumes of PBS and then protein was eluted with approximately 5 column volumes of 0.1 M glycine pH2.0. In this case (contrasting with the previous example), further purification was then undertaken. Protein (in tris-glycine) was buffer exchanged to 20 mM acetate pH 5.0 prior to loading using the Akta onto 1 (or 2 in parallel) 6 ml resource S columns (GE healthcare) pre-equilibrated in 20 mM acetate pH 5.0. After washing with the same buffer, protein was eluted via a 0-0.75 M or NaCl gradient in 20 mM acetate pH5.0. Fractions of the correct size were then identified by SDS-PAGE electrophoresis and by mass spectrometry and were then combined to make the final protein sample. Protein was then buffer exchanged into 20 mM citrate, pH6.2, 100 mM NaCl and concentrated to between 0.5 and 5 mg/ml. Protein was filtered through a 0.2 uM filter to ensure sterility.

### Example 5

### Production of the PYY (3-36) Dom7h-14-10 (R108C) AlbudAb Peptide Conjugate (Which has the Structure Shown in FIG. 3) and Which is: a Dom7h-14-10 (R108C) albudab Conjugated to the PYY3-36 via a Lysine and a 4 Repeat PEG Linker)

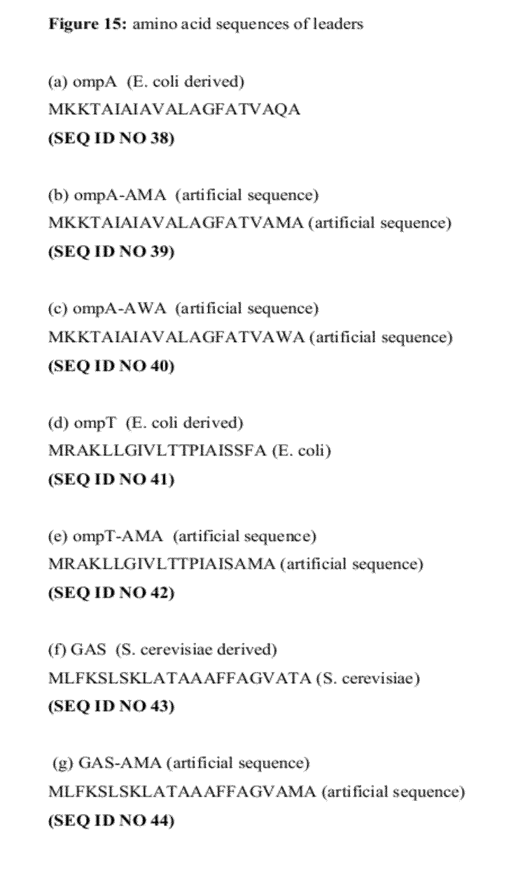

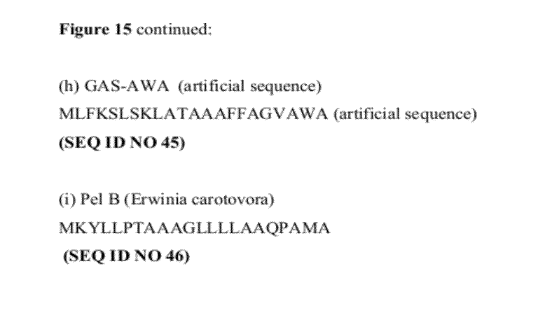

The Dom7h-14-10 (R108C) albudab was expressed and purified as described as follows in E. coli: The gene encoding the DOM7h-14-10 (R108C) was cloned into vector pET30. To enable cloning into expression vector, fusions were produced as assembly PCRs with NdeI restriction site on 5′ followed by the PEL B leader sequence (amino acid sequence shown in FIG. 15 (i) SEQ ID NO 46). Vector and assembly PCRs were digested with NdeI and BamHI restriction endonucleases followed by ligation of the insert into the vector using a Quick Ligation Kit (NEB). 2 microlitres of this ligation was used for transformation of MachI cells. After the recovery growth period, cells were plated on agar plates containing carbenicilin and incubated at 37° C. overnight. Colonies were sequenced and those containing the correct sequence were used for plasmid propagation and isolation (Plasmid Mini Prep kit, Qiagen). BL21(DE3) cells were transformed with plasmid DNA and resulting colonies were used for inoculation of expression culture. Expression was performed by inoculation of a 250 ml flask containing 50 ml of modified terrific broth media (Sigma) and this was inoculated at an OD=0.1 and was then grown at 30 deg C. supplemented with 50 mg/ml Kanamycin. At A600=0.5-1 cells were induced with IPTG to 50 uM final concentration, and growth was continued at 23 deg C. overnight. Then the culture supernatant was clarified by centrifugation at 3700×g for 1 hour. The expressed protein was then purified from the clarified supernatant using Protein L streamline (GE Healthcare, Cat. No. 28-4058-03, protein L coupled), and eluted from the Protein L using 0.1 M glycine pH2.0, then neutralized by addition of ⅕^{th} elution volume of 1 M Tris, pH8.0. The protein was then pH adjusted using 0.1 M Citric Acid to pH5 and applied to a 30 ml Source S column (GE Healthcare) equilibrated with 50 mM Sodium Citrate, pH5. A gradient from 0-100 of 50 mM Sodium Citrate, pH5, 1 M NaCl was applied using the AktaXpress FPLC (GE healthcare) over 150 ml. Fractions were analyzed on SDS-PAGE and those containing the purest product were pooled. The final protein was desalted into 50 mM Sodium Phosphate, pH6.5, 5 mM EDTA.

The Dom7h-14-10 (R108C) albudab was then linked to a PYY 3-36 amino acid molecule (but with a lysine at position 10 which can be derivatised with PEG linker) using the PEG linker shown in FIG. 3. The PYY and the PEG were prepared by standard chemical synthesis. The maleimide at the end of the PEG linker was then used to conjugate the PYY peptide to the free cysteine of the Dom7h-14-10 (R108C) albudab prepared as described above. The free cysteine of Dom7h-14-10 (R108C) was reduced by addition of Dithiothreitol (DTT) to a final concentration of 5 mM, incubated for 30 minutes and finally desalted into 50 mM Sodium Phosphate, pH6.5, 5 mM EDTA to remove the DTT. Maleimide activated peptide was then mixed with the protein at a 1:1 ratio and incubated to allow the conjugation to occur.

Conjugate was purified from un-reacted Dom7h-14-10 (R108C) by Ion Exchange chromatography in a similar manner to that described above. Fractions enriched in conjugate were finally purified from free peptide using Protein L affinity purification in a similar manner to described above. The final conjugate was buffer exchanged and analysed by SDS-PAGE and Mass Spectroscopy.

### Example 6

### Expression and Purification of Genetic Fusions of Exendin-4 and DOM7h-14-10/ DOM7h-11-15 AlbudAb

The aim of this experiment was to efficiently express DMS7139 and DMS7143. DMS7139 is a fusion of exendin-4 with DOM7h-14-10 (a domain antibody (dAb) that binds serum albumin, also known as an albudab) and DMS7143 is a fusion of exendin-4 with DOM 7h-11-15 (a domain antibody (dAb) that binds serum albumin, also known as an albudab) in E. coli with correctly processed N-termini. The fusion could then be tested for activity of the exendin-4 portion and of the AlbudAb portion in subsequent experiments. Exendin-4 was cloned as a fusion with DOM7h-14-10 or DOM7h-11-15, where exendin-4 peptide was at the 5′ end of the construct and AlbudAb at the 3′ end. In total two constructs were made each including (Gly4Ser)3 linker between the exendin-4 peptide and the AlbudAb. The linker was included as a spacer to separate the exendin 4 spatially from the dAb to prevent steric hindrance of the binding between the exendin-4 and the GLP-1 receptor. The sequences of the constructs are shown in FIGS. 1(m) and 1(n). To enable cloning into expression vector, fusions were produced as assembly PCRs with NdeI restriction site on 5′ followed by modified OmpT (OmpT AWA the amino acid sequence is shown in FIG. 1(q), SEQ ID NO 17) signal peptide and with BamHI site on 3′ terminus. OmpT AWA signal peptide has the last three codons changed from wildtype “TCTTTTGCC” to “GCTTGGGCC” which codes AWA instead of SFA. That change improves processing at the correct site by the signal peptidase of E. coli.

Additionally the sequence of the fusion starts straight after the peptidase cleavage site. An NcoI digestion site has been introduced, which overlaps with the last codon of the signal peptide and two first amino acids of exendin-4 sequence. This change facilitates future subcloning as well as leading to production of the fusion with free N-terminal end of exendin-4. The modified pET12a expression vector comprising the changes listed above was given the name pDOM35. Vector and assembly PCRs were digested with NdeI and BamHI restriction endonucleases followed by ligation of the insert into the vector using a Quick Ligation Kit (NEB). 2 microlitres of this ligation was used for transformation of MachI cells. After the recovery growth period, cells were plated on agar plates containing carbenicilin and incubated at 37° C. overnight. Colonies were sequenced and those containing the correct sequence were used for plasmid propagation and isolation (Plasmid Mini Prep kit, Qiagen). BL21(DE3) cells were transformed with plasmid DNA and resulting colonies were used for inoculation of expression culture. Expression was performed by inoculation of a 4×0.5 litre culture of TB Onex media (supplemented with Overnight Express™ autoinduction solutions), 1 droplet of antifoam (antifoam A204; Sigma) and 100 microgram per milliliter of carbenicillin. Culture was incubated for 3 nights at 30° C. with agitation 250 rpm, and then the culture supernatant was clarified by centrifugation at 3700×g for 1 hour. The expressed protein was then purified from the clarified supernatant using protein L streamline (GE Healthcare, Cat. No. 28-4058-03, protein L coupled), and eluted from the Protein L using 0.1 M glycine pH2.0, then neutralized using 0.1 volume of 1 M Tris pH8.0. Next protein was concentrated and dialysed to Buffer A (20 mM sodium acetate-acetic acid pH 5.0) and purified by Ion Exchange Chromatography on the AktaXpress (GE healthcare). Protein was loaded on Resource S 6 ml column in Buffer A (no salt buffer) and than eluted with Buffer B gradient (20 mM sodium acetate-acetic acid pH 5.0 1 M NaCl) from 0-75% B in 75 minutes in fractions. Fractions were analyzed on SDS-PAGE and by Mass Spectrometry and those of the correct mass were pooled. The final protein was dialyzed into 20 mM citrate 0.1 M NaCl buffer, and identity was reconfirmed by SDS-PAGE and Mass Spectrometry.

### Example 7

### Pharmacologic Profile of the Exendin-4 AlbudAb (DAT 0115 Made as Described Above) and PYY (3-36) AlbudAb Fusion Peptide (Made as Described in Example 5 and with the Structure Shown in FIG. 3) in the Melanophore Functional Bioassay

The pharmacologic profile of the Exendin-4 AlbudAb (DAT 0115) and the PYY(3-36) AlbudAb (as described in example 5 and with the structure shown in FIG. 3) was determined in a melanophore functional bioassay using cells transfected with receptors of interest. The bioassay was performed essentially as described in Jayawickreme et al. (2005) Current Protocols in Pharmacology 12.9.1-12.9.16.

The pharmacologic profiles of the Exendin-4 and PYY (3-36) AlbudAb fusion peptides are shown in Table 4. Results demonstrate that both Exendin-4 and PYY (3-36) fusion peptides retain the ability to activate both the human and mouse forms of their cognate receptors (Exendin-4 AlbudAb/GLP-1R and PYY (3-36)/NPY2R). The apparent selectivity of the PYY (3-36) AlbudAb for the NPY receptors ranks in the following order; NPY2R>NPY5R*>NPY1R>NPY4R for the human receptors and NPY2R>NPY5R>NPY4R>NPY1R for the mouse receptors. Selectivity values range from several hundred to >1000 fold, when comparing peptide activity for NPY2R to the other NPY receptors within the same species (calculated from Table 5).

TABLE 4
Peptide-Receptor pharmacologic profiles for Exendin-4
AlbudAb and PYY (3-36) AlbudAb fusion proteins

| | Human | | | Mouse | | |
|---|---|---|---|---|---|---|
| Receptor/Albudab | pEC50 | stdev | n | pEC50 | stdev | n |
| GLP1R/exendin-4 | 11.36 | 0.14 | 3 | 11.06 | 0.40 | 3 |
| NPY1R/PYY 3-36 | 7.33 | 0.27 | 4 | 7.13 | 0.22 | 4 |
| NPY2R/PYY 3-36 | 10.30 | 0.18 | 4 | 10.63 | 0.30 | 4 |
| NPY4R/PYY 3-36 | 6.91 | 0.43 | 4 | 7.71 | 0.59 | 4 |
| NPY5R/PYY 3-36 | nd | nd | nd | 8.30 | 0.46 | 4 |

### Example 8

### Exendin-albudab (DAT 0115) in Combination with PYY-albudab (as Described in example 5 and with the Structure Shown in FIG. 3) Causes Synergistic Effects on Multiple Parameters in Diet Induced Obese (DIO) Mice

Male diet induced obese (DIO) C57BL/6 mice (Taconic, Hudson, N.Y.) and lean C57BL/6 mice (Taconic, Hudson, N.Y.) were used for all experiments. DIO C57BL/6 mice were group housed and fed a high fat diet (45% fat by kcal) by the vendor from the time of weaning. DIO mice (40-50 g body weight) and age-matched controls were single-housed and maintained at constant temperature (approximately 22° C.) with 12 hr light/dark cycle (lights on from 5:00 AM to 5:00 PM). Mice were given ad libitum access to food (Research Diets D12451, 45% fat for DIO; Lab Diet 5001, 13.5% fat for lean) and water. All animal protocols were approved by the institutional animal care and use committee at GlaxoSmithKline in Research Triangle Park, N.C. The peptide-AlbudAbs were either prepared fresh daily or were prepared once and frozen at −70 deg C. in aliquots. For combination dosing, the drugs were mixed together so that only one injection would be required.

Chronic Obesity Efficacy Studies: DIO C56BL/6 mice and age-matched lean controls were habituated in house for 6 weeks before the start of the study. Animals were dosed every two days between 2-4 pm subcutaneously with a dose volume of 5 ml/kg over a period of 15 days.

Groups of Animals were dosed as follows:
(a) were given the PYY-albudab at 0.1 mg/kg (PYY ED20 GROUP)
(b) were given the PYY-albudab at 1.0 mg/kg (PYY ED80 GROUP)
(c) were given exendin-albudab (DAT 0115) at 0.01 mg/kg (Exendin ED20 GROUP)
(d) were given exendin-albudab (DAT 0115) at 0.1 mg/kg (Exendin ED80 GROUP)
(e) ED 20 combo: were given a single dose of: the PYY-albudab at 0.1 mg/kg mixed with the exendin-4-albudab (DAT 0115) at 0.01 mg/kg
(f) ED 80 combo: were given a single dose of: the PYY-albudab at 1.0 mg/kg mixed with the exendin-4-albudab (DAT 0115) at 0.1 mg/kg
(g) Control Exendin-4 alone given at 0.1 mg/kg.

A three day vehicle lead in period was used before the start of drug with the first day being vehicle and the second two days being mock injections. Baseline fat mass and lean mass measurements were taken 3-4 days before the start of drug and on day 15 using a QMR instrument (Echo Medical Systems, Houston, Tex.) Body weight measurements were taken every Monday, Wednesday, and Friday starting four days before the first drug dose, with the first measurement being used to randomize the animals. Food hopper weights were measured every weekday starting 4-6 days before the first drug dose, allowing for the calculation of food intake. Animals that created excessive food spillage were removed prior to the beginning of the study. During the study, excess food was removed from the cage and added to the food hopper weights for increased accuracy. Eight to ten animals (n=8-10) were used for the lean control group and eight animals (n=8) were used for all other treatment groups. Sixteen days after the start of drug treatment, animals were fasted for at least 4 hours before collection of whole blood, plasma, and serum samples via terminal cardiac exsanguinations. The whole blood was used to determine the % HbAlc, the plasma was used for a gastrointestinal hormone panel, and the serum was used to access multiple clinical chemistry parameters. Finally, major organs and tissues were collected (heart, kidney, liver, lung, stomach, duodenum, colon, pancreas, brown adipose, white adipose, carcass) on day 16 and fixed in 10% neutral buffered formalin for macroscopic and microscopic histological examination.

A) Effect of Exendin-4-albudab (DAT 0115) in Combination with PYY-albudab on Body Weight

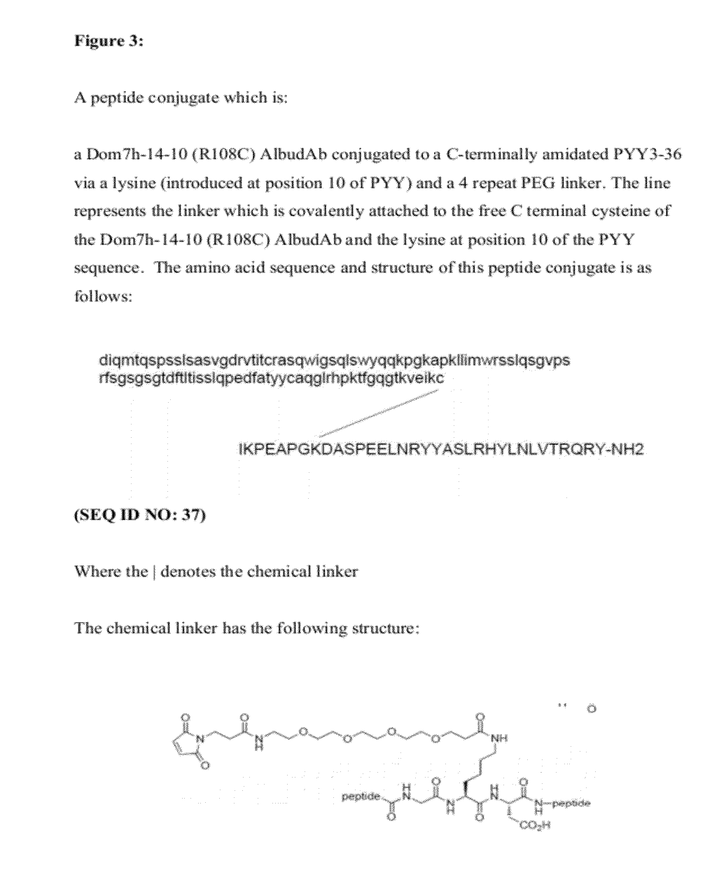

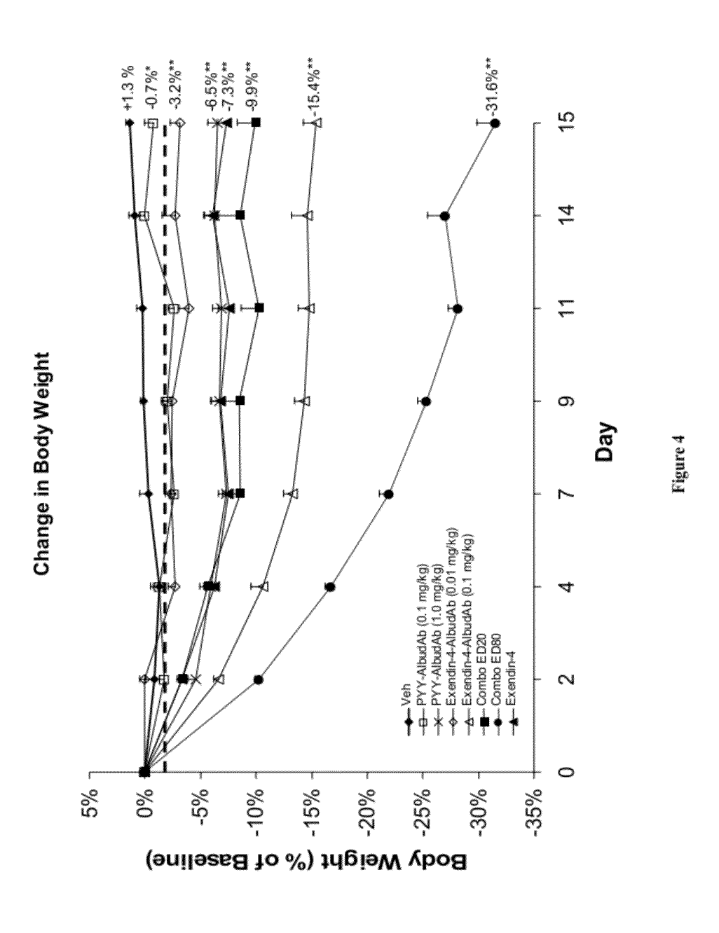

All the treatment groups described above demonstrated clear and sustained decreases in body weight. See FIG. 4. The effects generally plateaued after 7 days for all treatment groups except the Combo ED₈₀. The Combo ED₈₀ did not reach a plateau by 15 days of treatment. At day 15, the addition of the PYY-AlbudAb 0.1 mg/kg dose (2% decrease vs. vehicle) plus the Exendin-4-AlbudAb 0.01 mg/kg dose (4.5% decrease vs. vehicle) indicates that a 6.5% decrease in body weight relative to vehicle control would be expected. However, an 11.2% decrease in body was the observed weight when the AlbudAbs were combined in the Combo ED₂₀ group, which is greater than the expected additivity (p<0.05).

For the ED₈₀ group a greater than additive effect on body weight was observed only after the first 7 days of treatment. If the effects of these treatments were additive at day 7, then a 20.1% decrease in body weight relative to vehicle (7.1% for PYY-AlbudAb 1.0 mg/kg and 13.0% for Exendin-4-AlbudAb 0.1 mg/kg) would be expected. For the Combo ED₈₀ group at day 7, a 21.6% decrease was observed which is not statistically significant from the predicted additivity data. However, at the 15 day time point, the PYY-AlbudAb 1.0 mg/kg group showed about a 7.8% decrease from vehicle and the Exendin-4-AlbudAb 0.1 mg/kg group showed a 16.8% decrease from vehicle; addition of those two dose groups would have yielded a 24.6% decrease in body weight. In fact, a 32.8% decrease for the Combo ED₈₀ group was observed which is a statistically significant increase over the predicted additivity data (p<0.05).

B) Effect of Exendin-4-albudab (DAT 0115) in Combination with PYY-albudab on Change in Food Intake

Some level of inhibition of food intake was observed for all of the treatment groups relative to vehicle controls. See FIG. 5. All treatment groups except the Combo ED₈₀ group reverted back to vehicle control levels over time. For days 1 and 2, the Combo ED₂₀ showed a daily average 25.1% inhibition of food intake from baseline (normalized to vehicle), although addition of the two groups would have predicted a modest decrease of 5.7% in food intake. At all other time points, an additive effect was observed.

For the ED₈₀ dose groups (PYY-AlbudAb 1.0 mg/kg and Exendin-4-AlbudAb 0.1 mg/kg) an additive effect on weight was observed during the early time points. However, starting at the day 10 time point, a 42% inhibition in food intake was observed while a 17% inhibition of food intake would be predicted if the effect of the combination was merely additive(p<0.05). This effect continued for the remainder of the study and may be best exemplified at day 14 where the addition of the PYY-AlbudAb 1.0 mg/kg group (2.5% inhibition of feeding) and the Exendin-4-AlbudAb 0.1 mg/kg group (0.8% inhibition of feeding) predicts a 3.3% inhibition of food intake for the combination of the two groups (Combo ED₈₀). Ultimately, a 19.2% inhibition of food intake was observed in the Combo ED₈₀, which is a statistically significant difference (p<0.05) from what would be predicted if the combination had an additive effect. The inhibition of food intake in the combination groups indicates that anorectic activity accounts for at least part of the mechanism of weight loss for the combination of PYY-AlbudAb and Exendin-4-AlbudAb.

C. Effect of Exendin-4-albudab (DAT 0115) in Combination with PYY-albudab on Change in Body Composition

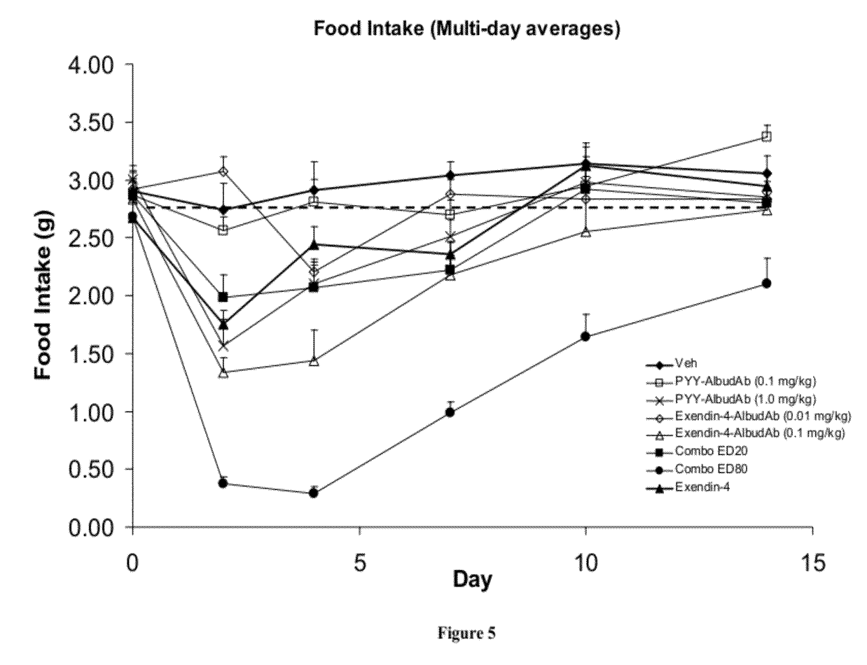

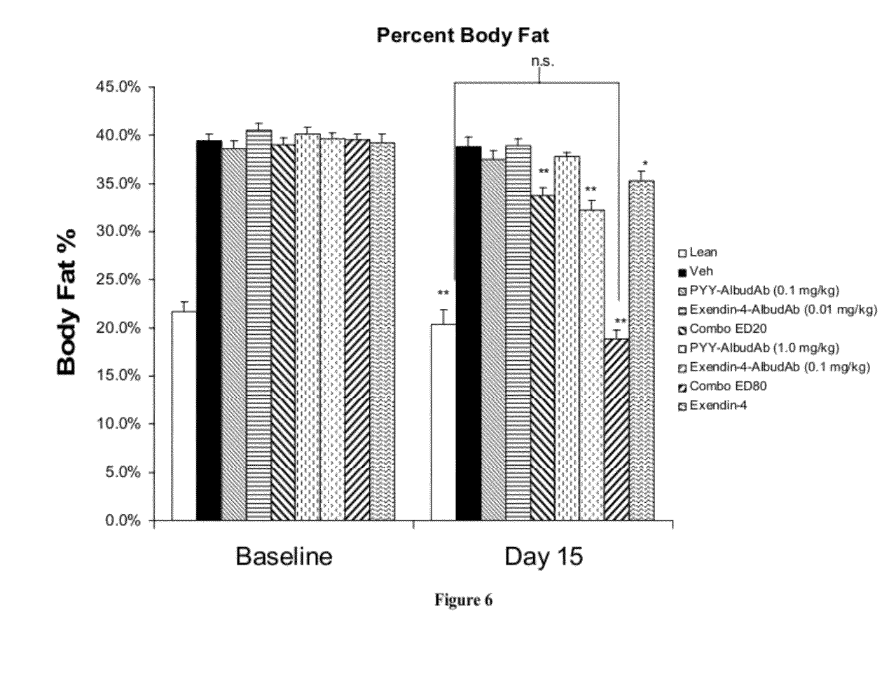

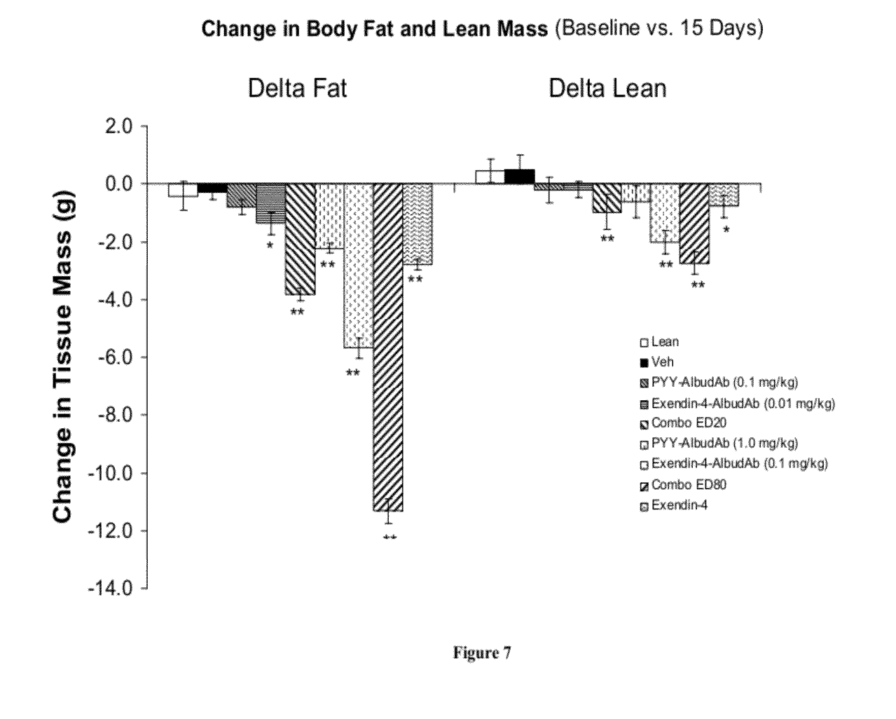

Absolute changes in percent body fat were observed for the Exendin-4 AlbudAb 0.1 mg/kg group, the Combo ED₂₀ group, and the Combo ED₈₀ group (p<0.01 vs. vehicle for all groups). See FIGS. 6 and 7. Both of the Combo treatments groups also demonstrated a decrease in body fat percent over the 15 day treatment period that was consistent with a greater than additive effect of the combination. Specifically, the percent body fat of the PYY-AlbudAb 0.1 mg/kg group dropped by 1.8% and the Exendin-4-AlbudAb 0.01 mg/kg group showed a 0.6% decrease in body fat, neither of which represents a significant change (both values normalized to changes in vehicle controls). In contrast, for the Combo ED₂₀ treatment group, there was a 4.8% decrease in percent body fat which is significantly more than the predicted additive value of 2.4% (p<0.05). For the higher doses, the predicted additive decrease would be 8.6% (PYY-AlbudAb 1.0 mg/kg and Exendin-4-AlbudAb 0.1 mg/kg; decrease of 1.8% and 6.8% respectively). However, the observed change in the Combo ED₈₀ group was a 20.0% decrease, which is significantly greater than what was predicted by additivity (p<0.05).

The Combo ED₈₀ group dropped from 39.5% body fat down to 18.9% body fat. There was no longer a significant difference in percent body fat between the lean controls and the Combo ED₈₀ (p=0.43). Therefore, the Combo ED₈₀ group was “normalized” back to lean control, despite being maintained in an obesity-prone environment (i.e. access to a high-fat diet). This corresponds to a 100% loss of

excess body fat.

A dose-dependant change in fat mass was observed for both the monotherapies and combination treatment groups. During the treatment period, the PYY-AlbudAb 0.1 mg/kg group lost 0.8 grams of fat mass (p=0.29 vs. vehicle control) while the Exendin-4-AlbudAb group lost 1.4 grams of fat mass (p<0.05 vs. vehicle control). If these treatments had an additive effect on fat mass, we would expect the Combo ED₂₀ group to lose 2.2 grams of fat mass. However, the Combo ED₂₀ group lost 3.8 grams of fat mass which is significantly greater than the predicted additivity value (p<0.05).

A similar analysis was conducted for the ED₈₀ dose group. The PYY-AlbudAb 1.0 mg/kg group lost 2.2 grams of body fat (p<0.01 vs. vehicle control) while the Exendin-4-AlbudAb group lost an average of 5.7 grams of body fat (p<0.01 vs. vehicle control). The addition of these two groups would suggest that in combination, a 7.9 gram loss of body fat would be predicted. However, a loss of 11.3 grams of body fat for the Combo ED₈₀ group (p<0.01 vs. vehicle control) was observed. The difference between the expected data based on additivity and the observed data is statistically significant (p<0.05).

Although some lean mass loss was observed among the treatment groups, the magnitude of the effect was much smaller on lean mass than on fat mass. Overall, approximately 80% of all weight lost was fat mass, which is consistent with ratio of fat mass vs. lean mass loss observed in clinical trials using dieting and exercise.

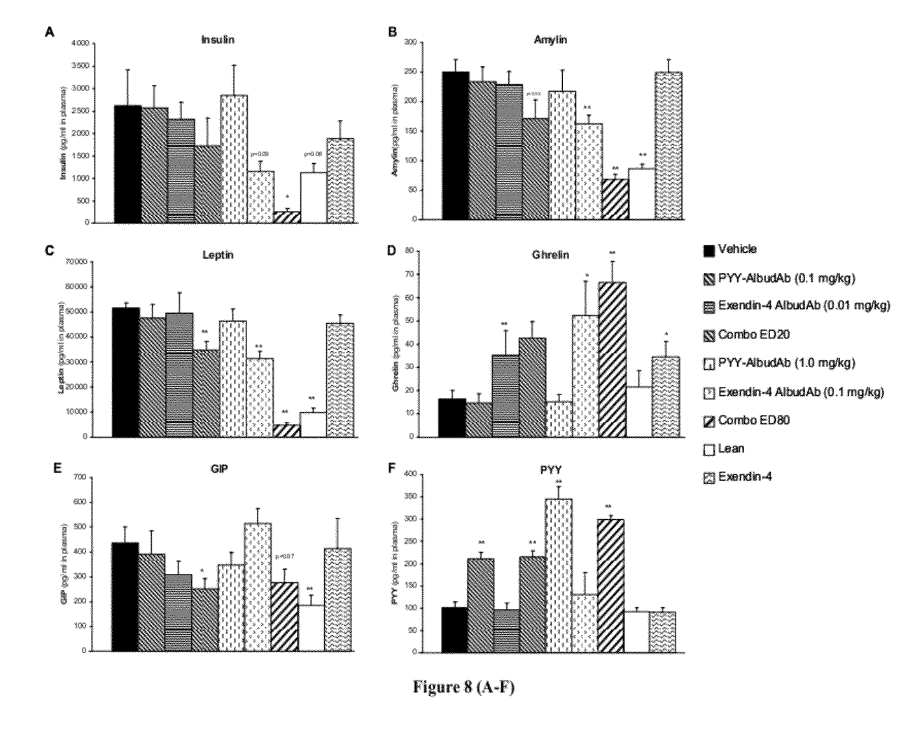

D. Effect of Exendin-4-albudab (DAT 0115) in Combination with PYY-albudab on Change in Endocrine Analytes (see FIG. 8)

For the Combo ED₈₀ group, insulin levels were only 1/10^{th} of the vehicle control levels (2617 pg/ml and 259 pg/ml in plasma respectively, p<0.05). This decrease in insulin is logical because the animals were normoglycemic at the beginning and end of the study. That is, the decreased insulin is presumably protecting against hypoglycemia.

Leptin levels in the combo ED₈₀ group were lower than the vehicle control group by over 90% (51.6 ng/ml in plasma for vehicle; 4.7 ng/ml in plasma for Combo ED₈₀, p<0.01). This was comparable to the lean control levels (9.8 ng/ml in plasma) which is likely due to the dramatic decrease in fat mass in the Combo ED₈₀ group. In addition, the Combo ED₂₀ and the Exendin-4-AlbudAb 0.1 mg/kg groups had plasma leptin values that were significantly lower than the vehicle controls (34.8 ng/ml, p<0.01 and 31.4 ng/ml, p<0.01 respectively). These effects appear to be related to the decrease in fat mass.

Gastric Inhibitory Peptide (GIP) levels were decreased significantly in the Combo ED₂₀ (p<0.05 vs. vehicle control) and showed a strong trend in the Combo ED₈₀ group (p=0.08 vs. vehicle control).

Amylin levels in the Combo ED₈₀ group (68 pg/ml in plasma) were significantly lower than the vehicle controls (250 pg/ml in plasma; p<0.01). Moreover, the Combo ED₈₀ amylin levels were approximately the same as the lean control levels (87 pg/ml in plasma). The Combo ED₂₀ group showed a strong trend toward a decrease (171 pg/ml in plasma; p=0.054 vs. vehicle control) and the Exendin-4-AlbudAb 0.1 mg/kg group was significantly lower than vehicle control (163 pg/ml in plasma; p<0.01).

Ghrelin levels were elevated in the Exendin-4-AlbudAb monotherapy groups to a level approximately equal to the combination groups. This indicates that Exendin-4 activity alone is most likely responsible for the increased ghrelin exposure.

PYY levels were elevated in animals receiving PYY-AlbudAb, probably due to direct detection of the dosed peptide in plasma. These values however are not indicative of absolute levels of PYY-AlbudAb in circulation.

E. Effect of Exendin-4-albudab (DAT 0115) in Combination with PYY-albudab on Changes in Serum Chemistry Parameters

Overall, there was an excellent profile observed for serum chemistries in most treatment groups which included the Combo ED₂₀ and all groups tested at ED₈₀. The Lean Control group represents the relative difference between lean animals and the DIO group. Values represent changes for all other groups because these groups were randomized from a single population prior to the beginning of the study. The Combo ED₂₀ group displayed some significant improvements on glucose and total cholesterol, while showing trends towards improvements in triglycerides and alanine transaminase (ALT) levels (Table 5).

Significant improvements were observed for the PYY-AlbudAb 1.0 mg/kg group and the Exendin-4-AlbudAb 0.1 mg/kg group in the areas of lowering glucose, total cholesterol, total bilirubin, creatinine, aspartate aminotransferase (AST), alanine transaminase (ALT), and total protein. However, these effects were generally to a lesser extent than what was observed in combination (Combo ED₈₀). The Combo ED₈₀ group displayed many significant changes in serum chemistries. All of these changes (with the exception of blood urea nitrogen (BUN)) represent improvements that moved the animal from the pathological state of obesity to the normal lean state. For example, the liver enzyme alanine transaminase (ALT) is elevated in the vehicle control DIO mice but treatment with the Combo ED₈₀ decreased levels by 79% to the level of the lean controls. Other significant improvements include HbAlc, total cholesterol, triglycerides, total bilirubin, creatinine, aspartate aminotransferase (AST), alanine transaminase (ALT) and total protein. All of these changes made the DIO serum chemistries more closely resemble the lean control chemistries and were considered beneficial.

TABLE 5
Summary of Serum Chemistry Parameters

| | % Change from DIO Vehicle | | | | | | | |
|---|---|---|---|---|---|---|---|---|
| | ED20 Doses | | | ED80 Doses | | | Controls | |
| | PYY-Alb | Exn-Alb | | PYY-Alb | Exn-Alb | | | Exenatide |
| Parameter | (0.1 mg/kg) | (0.01 mg/kg) | Combo | (1.0 mg/kg) | (0.1 mg/kg) | Combo | Lean | (0.1 mg/kg) |
| HbA1c | — | — | — | — | — | ↓-4%* | ↓-9%* | — |
| Glucose | — | — | ↓-10%* | ↓-13%* | ↓-27%* | ↓-27%* | ↓-12% | ↓-13%* |
| Insulin | | | ↓-34% | | ↓-56% | ↓-90%* | ↓-57% | |
| Total Cholesterol | — | — | ↓-16%* | — | ↓-24%* | ↓-49%* | ↓-67%* | ↓-11%* |
| Triglycerides | — | — | ↓-16% | — | — | ↓-24%* | ↓-41%* | — |
| Total Bilirubin | — | — | — | ↑-26% | ↑21%* | ↑49%* | ↑-12% | ↑26%* |
| β-hydroxybutyrate | — | — | — | ↓-38%* | — | — | — | ↓-41%* |
| Blood Urea Nitrogen | — | — | — | — | — | ↓-22%* | ↑27%* | — |
| Creatinine | — | — | — | — | ↓-17%* | ↓-21%* | ↓-16%* | — |
| AST | — | — | — | — | ↓-41%* | ↓-50%* | ↓-25% | ↓-25% |
| ALT | — | — | ↓-29% | ↓-30% | ↓-57%* | ↓-79%* | ↓-72%* | ↓-41% |
| Total Protein | — | — | — | ↓-4%* | — | ↓-8%* | ↓-9%* | — |

↓↑ Bold* = P < 0.05
↓↑ = trend

F. Effect of Exendin-4-albudab (DAT 0115) in Combination with PYY-albudab on Changes in Histopathology

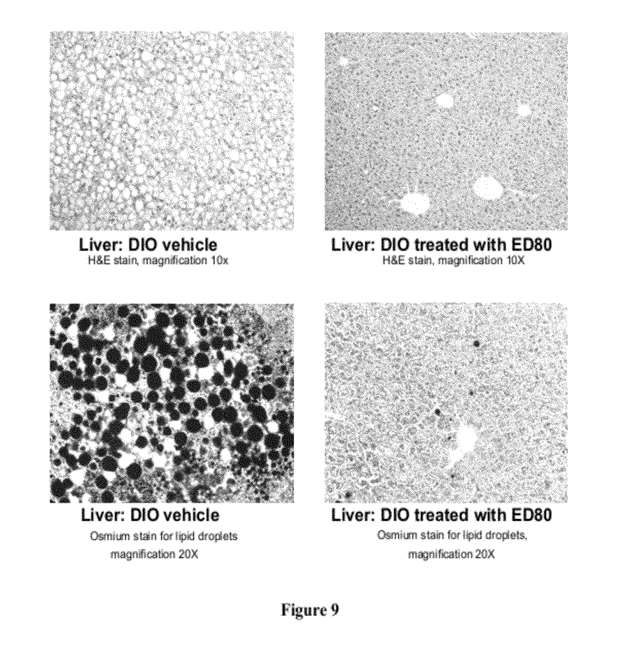

Cytoplasmic lipid droplets in the liver, confirmed by osmium stain, were marked in severity in the DIO vehicle-control mice, affecting most hepatocytes. The cytoplasmic lipid droplets were substantially decreased (minimal to undetectable) in DIO mice given Combo ED₈₀ (see FIG. 9). A similar change with lesser response magnitude than seen in Combo ED₈₀ livers was noted in DIO mice given Combo ED₂₀, PYY-AlbudAb (1.0 mg/kg), Exendin-4-AlbudAb (0.1 mg/kg) and Exendin-4 (0.1 mg/kg). However, a test article-related microscopic change, consisting of decreased cytoplasmic lipid droplets was observed in the liver [Combo ED₂₀, Combo ED₈₀, PYY-AlbudAb (1.0 mg/kg), Exendin-4-AlbudAb (0.1 mg/kg) and Exendin-4 (0.1 mg/kg)], brown adipose tissue [Combo ED₂₀, Combo ED₈₀, PYY-AlbudAb (1.0 mg/kg), Exendin-4-AlbudAb (0.01- and 0.1 mg/kg) and Exendin-4 (0.1 mg/kg)] and kidney (only in Combo ED₈₀) of treated DIO mice. These tissue changes in these groups correlated with decreases in serum transaminases, total cholesterol, HDL, and glucose. Combo groups ED₂₀ and ED₈₀ also had decreased triglycerides. These changes were related to the intended pharmacology and considered beneficial.

### Example 9

### Effects of Exendin-AlbudAb (DAT 0115) and PYY-Albudab (as Described in Example 5 and with the Structure Shown in FIG. 3) Combination on Diabetes Parameters in db/db Mice

Male db/db C57BL/6J mice (Jackson Labs, Bar Harbor, Me.) were used for all experiments. The db/db mice (B6.Cg-m +/+ Leprdb/J) and controls were group-housed by the vendor. The db/db mice (10-12 weeks of age), and age-matched controls were shipped to GSK where they were single-housed and maintained at constant temperature (approximately 22° C.) with 12 hr light/dark cycle (lights on from 5:00 AM to 5:00 PM). Mice were given ad libitum access to food (LabDiet 5K67, 16% fat for db/db and their controls) and water. All animal protocols were approved by the institutional animal care and use committee at GlaxoSmithKline in Research Triangle Park, N.C. The peptide-AlbudAbs were prepared fresh daily. The correct dosing concentration of the drug was obtained by diluting the master stock using a citrate vehicle buffer comprised of 100 mM NaCl, 20 mM citric acid, pH 6.2 (filter sterilized). For combination dosing, the drugs were mixed together so that only one injection would be required.

Chronic Diabetes Efficacy Studies: The db/db mice and age-matched lean controls were habituated in house 2 weeks before the start of the study. Animals were dosed every two days between 2-4 pm subcutaneously with a dose volume of 5 ml/kg over a period of 15 days. A three day vehicle lead in period was used before the start of drug with the first day being vehicle and the second two days being mock injections. Baseline fat mass and lean mass measurements were taken 3 days before the start of drug and on day 15 using a QMR instrument (Echo Medical Systems, Houston, Tex.) Body weight measurements were taken every Monday, Wednesday, and Friday starting four days before the first drug dose. Blood samples were taken via tail snip to measure fed glucose values and % HbAlc values two days before the start of drug dosing; this data was used to randomize the animals into different groups. Food hopper weights were measured every weekday starting 4-6 days before the first drug dose, allowing for the calculation of food intake. Animals that created excessive food spillage were removed prior to the beginning of the study. During the study, excess food was removed from the cage and added to the food hopper weights for increased accuracy. Eight animals (n=8) were used for the lean control group and eight animals (n=8) were used for all other treatment groups. A pair-fed control was included in which the daily food intake for the combination ED₈₀ group was calculated and that amount of food was given to the pair-fed group to eat the next day. Sixteen days after the start of drug treatment, animals were fasted for at least 4 hours before collection of whole blood, plasma, and serum samples via terminal cardiac exsanguinations. The whole blood was used to determine the % HbAlc, the plasma was used for a gastrointestinal hormone panel, and the serum was used to access multiple chemistries. Finally, major organs and tissues were collected (heart, kidney, liver, lung, stomach, duodenum, colon, pancreas, brown adipose, white adipose, carcass) on day 16 and fixed in 10% neutral buffered formalin for macroscopic and microscopic histological examination.

A. Effect of Exendin-4-albudab (DAT 0115) in Combination with PYY-albudab on Changes in Percent Hemoglobin Alc

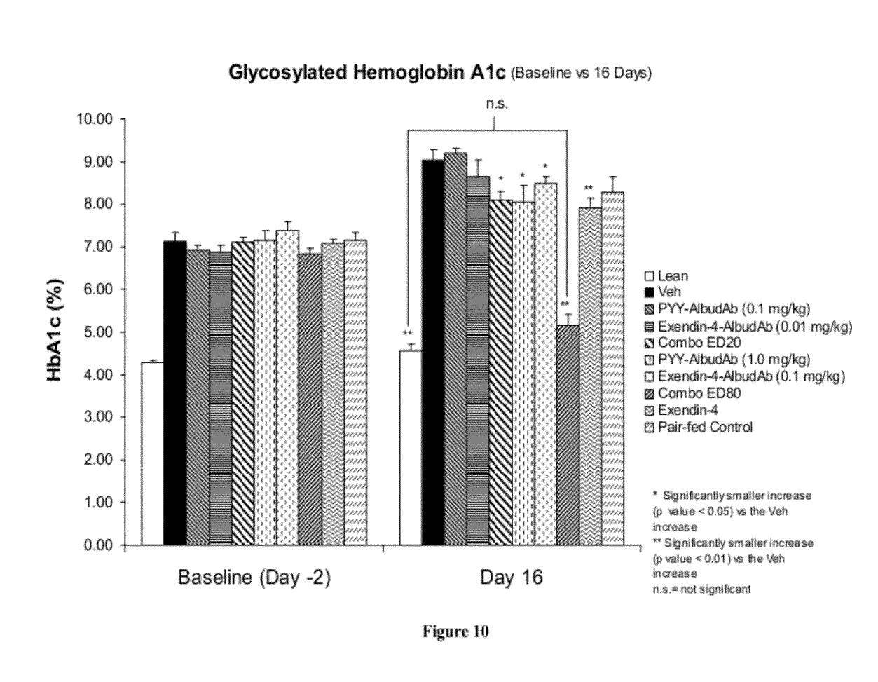

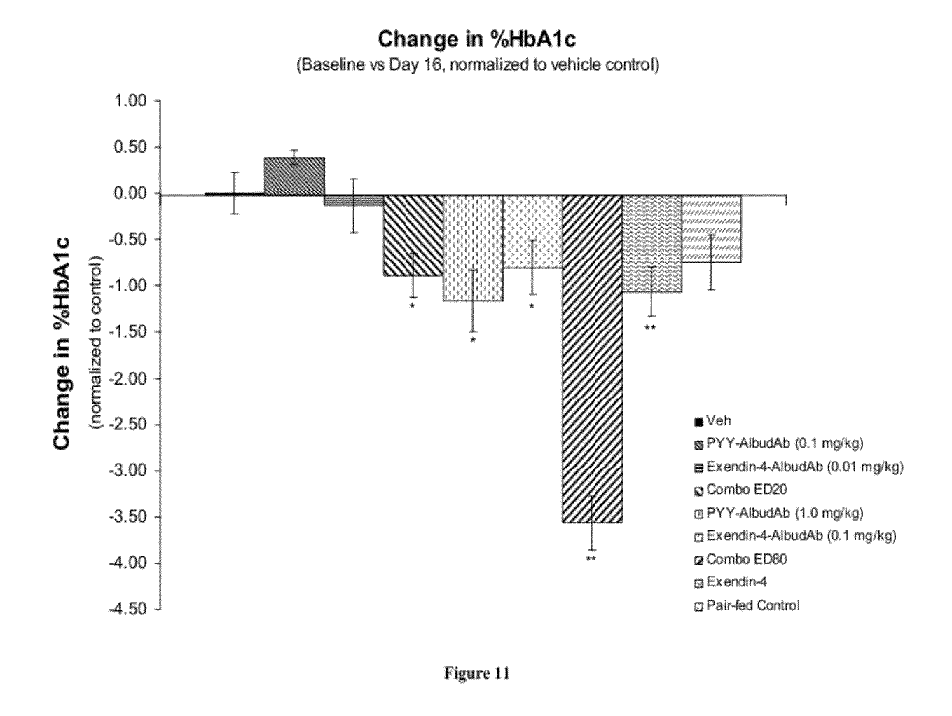

The vehicle control animals increased % HbAlc during the 18 days of the study from an average of 7.14% at baseline to an average of 9.03% by day 16. This indicates substantial progression of the diabetic phenotype during that time period. See FIGS. 10 and 11. An inhibition of the progression of the diabetic phenotype was observed in multiple dose groups including the Combo ED₂₀, the PYY-AlbudAb 1.0 mg/kg, and the Exendin-4-AlbudAb 0.1 mg/kg groups (p<0.05 vs. vehicle increase). An absolute decrease in % HbAlc was only observed for the Combo ED₈₀ group (p<0.01 vs. baseline). The Combo ED₈₀ group dropped from 6.83% glycosylated HbAlc down to 5.16% glycosylated HbAlc. There was no longer a significant difference in glycosylated HbAlc between the lean non-diabetic controls and the Combo ED₈₀ (p<0.01). Therefore, the diabetic (db/db) mice in the Combo ED₈₀ treatment group had a completely normal level of % glycosylated HbAlc and were nearly “normalized” back to normal lean control animals.

The Pair-fed Controls (fed the same amount of food as the Combo ED₈₀ animals consumed) showed no significant change from the vehicle control animals (p=0.11). This indicates that inhibition of food intake was not a major mechanism for HbAlc lowering of the Combo ED₈₀ group.

Significant changes in glycosylated hemoglobin were observed in multiple groups including the PYY-AlbudAb 1.0 mg/kg group (1.16% decrease, p<0.05), the Exendin-4-AlbudAb 0.1 mg/kg group (0.80% decrease, p<0.05) as well as in the Combo ED₂₀ group (0.89% decrease, p<0.05) and the Combo ED₈₀ group (3.57% decrease, p<0.01).

The Combo groups were analyzed in a similar manner. The PYY-AlbudAb 0.1 mg/kg group and the Exendin-4-AlbudAb 0.01 mg/kg groups showed no significant changes from the vehicle control levels while in combination (Combo ED₂₀), there was a 0.89% decrease in glycosylated HbAlc. For the ED₈₀ dose groups, the predicted additive decrease would be 1.96% for the PYY-AlbudAb 1.0 mg/kg and Exendin-4-AlbudAb 0.1 mg/kg groups. However, in the combination (Combo ED₈₀ group) a 3.57% decrease in glycosylated HbAlc was observed. This decrease is significantly greater than what was predicted by additivity of the monotherapy groups (p<0.05).

B. Effect of Exendin-4-albudab (DAT 0115) in Combination with PYY-albudab on Changes in Plasma Insulin

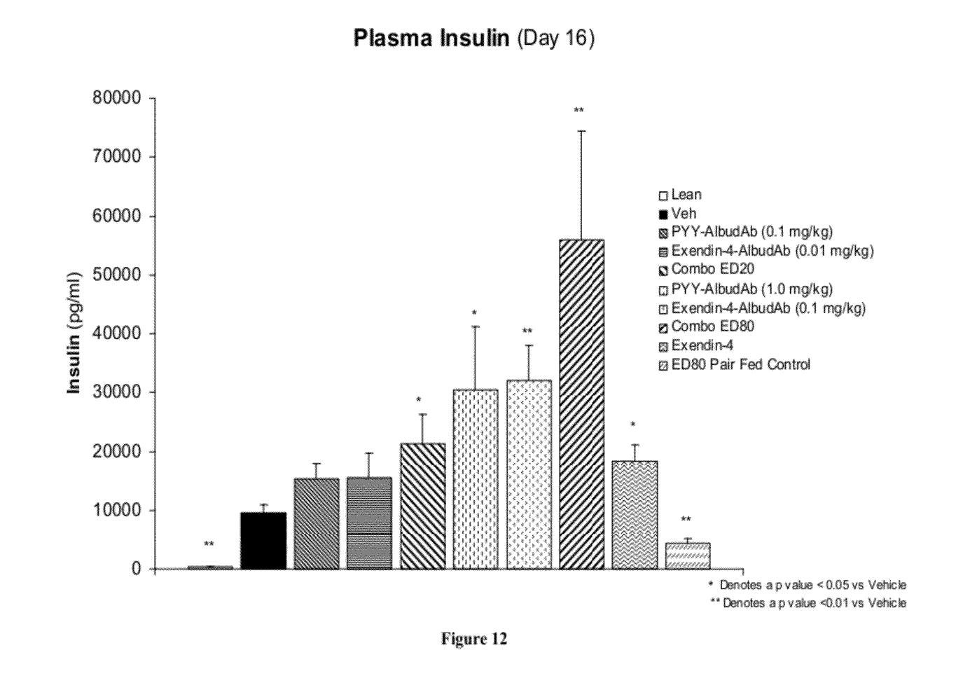

The low dose monotherapy treatment groups showed trends towards increases in plasma insulin levels when compared to the vehicle controls (PYY-AlbudAb 0.1 mg/kg, p=0.052; Exendin-4-AlbudAb 0.01 mg/kg, p=0.17). For the Combo ED₂₀ group, plasma insulin levels reached 21307 pg/ml which was significantly higher than the vehicle control group at 9470 pg/ml in plasma (p<0.05). The PYY-AlbudAb 1.0 mg/kg group (30467 pg/ml; p<0.05 vs. vehicle control) and the Exendin-4-AlbudAb group (32036 pg/ml; p<0.01 vs. vehicle control) also had elevated insulin levels. (See FIG. 12)

In the Combo ED₈₀ group, insulin levels were over 5 times higher than the vehicle control levels. (55950 pg/ml and 9470 pg/ml in plasma respectively, p<0.05). These exceptionally high levels of insulin are thought to be responsible for at least part of the glucose lowering effects observed in these animals.

The ED₈₀ Pair-fed Control group had plasma insulin levels of 4438 pg/ml which was significantly lower than the vehicle control levels (p<0.01), most likely due to the weight loss.

C. Effect of Exendin-4-albudab (DAT 0115) in Combination with PYY-albudab on Inhibition of Weight Gain

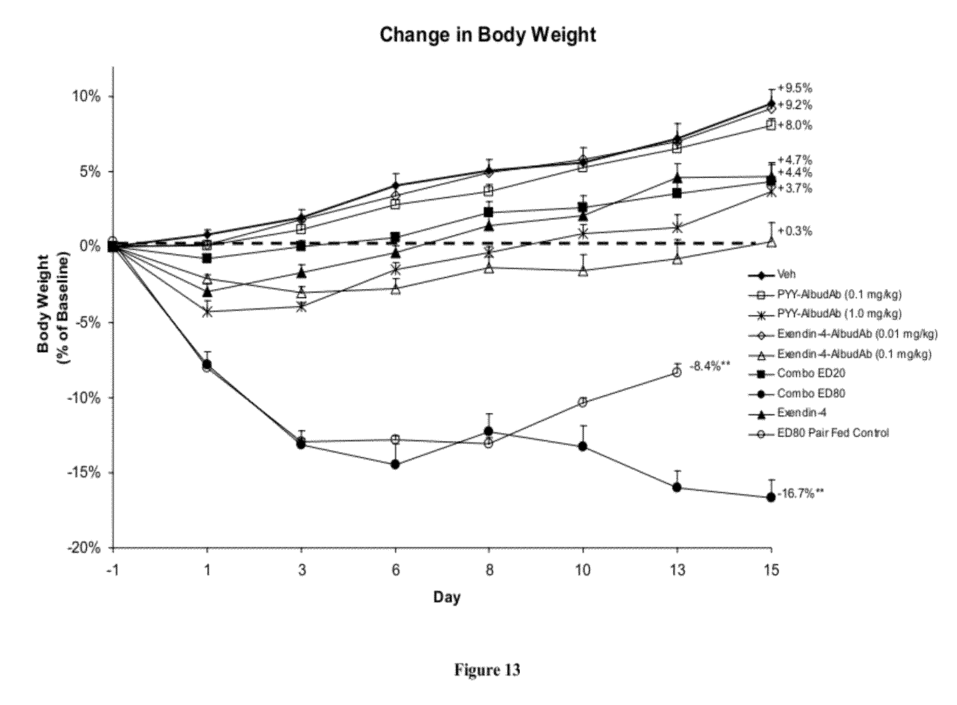

Body weight was also monitored for the diabetes study. Due to the rapid weight gain of db/db mice, this model can be used to assess inhibition of weight gain in addition to loss of body weight. This study indicates that the PYY-AlbudAb 1.0 mg/kg, the Exendin-4-AlbudAb 0.1 mg/kg, the Combo ED₂₀, and the Combo ED₈₀ treatments were effective at inhibiting weight gain. See FIG. 13.

By day 15, a clear collaboration had emerged between the PYY-AlbudAb 0.1 mg/kg which trended toward a 1.5% decrease relative to vehicle control (p=0.18) and the Exendin-4-AlbudAb 0.01 mg/kg which had no significant effect alone. In combination, the Combo ED₂₀ group gained significantly less weight than the vehicle controls (9.5% weight gain for vehicle, 4.4% weight gain for Combo ED₂₀; p<0.01).

The Combo ED₈₀ group was analyzed in a similar manner. At day 15, the PYY-AlbudAb 1.0 mg/kg group showed a 5.9% decrease from vehicle and the Exendin-4-AlbudAb 0.1 mg/kg group showed a 9.2% decrease from vehicle; addition of those two dose groups would have yielded a 15.1% decrease in body weight. In fact, a 26.2% decrease for the Combo ED₈₀ group was observed, which is a statistically significant increase over the predicted additivity data (p<0.05).

Over the first eight days, the Pair-fed Confrols (pair-fed to Combo ED₈₀ group) demonstrated a 12.8% loss in body weight that was comparable to the Combo ED₈₀ group (12.3% weight loss) over the same time period. However, after eight days the Pair-fed Controls gained weight at about the same rate as the vehicle controls, while the Combo ED₈₀ group maintained their weight loss. This resulted in a net weight loss of 8.4% for the pair-fed group and 16.7% for the Combo ED₈₀ group (p<0.01 vs. baseline for both groups). This rebound effect and resulting differences in body weight at day 15 suggests that a difference in metabolism is emerging between the pair-fed group and the Combo ED₈₀ group after eight days that is attributable to the combination and not merely to effects on weight.

D. Effect of Exendin-4-albudab (DAT 0115) in Combination with PYY-albudab on Inhibition of Food Intake

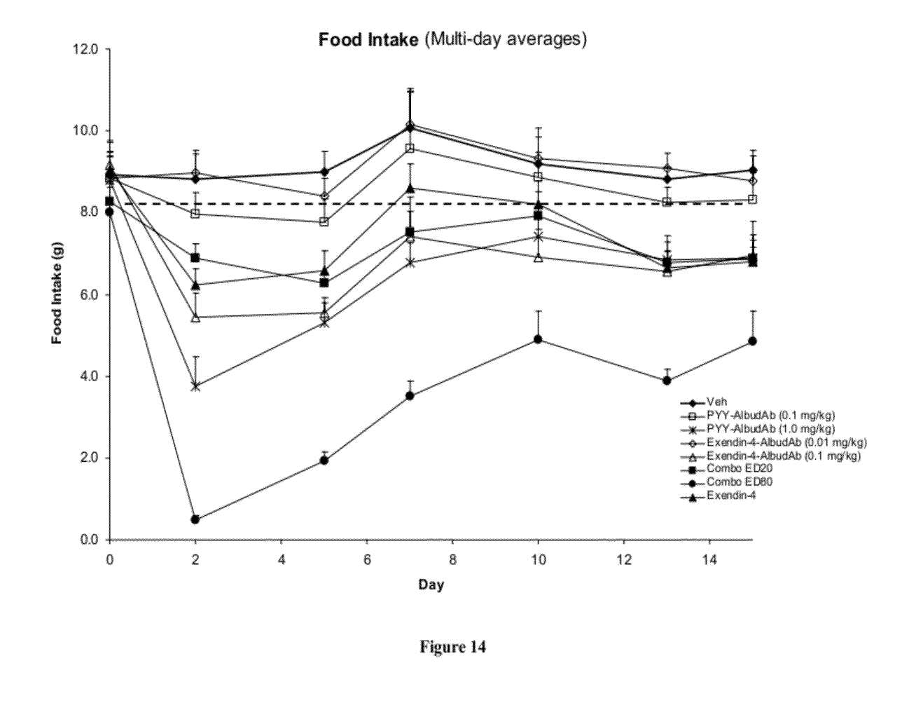

Significant decreases in food intake were observed over a fifteen day period in all groups except for the PYY-AlbudAb 0.1 mg/kg and the Exendin-4-AlbudAb 0.01 mg/kg groups. See FIG. 14. Generally, the inhibition of food intake was greater during the first five days, after which time there was somewhat of a stabilization of daily food intake. At day 15 (average of days 13-15), the Combo ED₂₀, PYY-AlbudAb 1.0 mg/kg, and the Exendin-4-AlbudAb 0.1 mg/kg groups all averaged 6.9 to 7.0 grams of food intake per day. This was significantly lower than the 9.0 grams of food consumed by the vehicle control group (p<0.05).

A dramatic decrease in food intake was initially observed for the Combo ED₈₀ group. Through day 5, animals in this group averaged less than 2 grams of food intake per day which is much less than 9 grams for the vehicle control animals (p<0.01). There was a small rebound in food intake observed through day 10, at which time the food intake levels stabilized. By day 15, the Combo ED₈₀ group was consuming 4.8 grams of food per day which is approximately half of the food intake of the vehicle control group.

Food intake did not rebound back to vehicle control levels in any of the groups where we observed a significant decrease in feeding. The food intake in the treatment groups stabilized and was approximately parallel to the vehicle control group from days 10 to 15 of the study. This suggests that these animals may remain in a negative energy balance (assuming no metabolic compensation) and that body weight may continue to decrease relative to vehicle controls.

### Example 10

### PYY3-36 AlbudAb (DMS7620) Dose-Dependently Suppresses Food Intake and Causes Weight Loss in Diet Induced Obese (DIO) Mice

Male diet induced obese (DIO) C57BL/6 mice (Taconic, Hudson, N.Y.) were used for all experiments. DIO mice were single-housed and maintained at constant temperature and humidity (approximately 22° C. and 50% respectively) with 12 hr light/dark cycle (lights on from 5:00 AM to 5:00 PM). Mice were given ad libitum access to food (Research Diets D12451, 45% fat for DIO) and water. All animal protocols were approved by the institutional animal care and use committee at GlaxoSmithKline in Research Triangle Park, N.C. The peptide- AlbudAbs were prepared once and frozen at −80 deg C. in daily aliquots. For combination dosing, the drugs were mixed together so that only one injection would be required.

Chronic Obesity Efficacy Studies: DIO C56BL/6 mice were habituated in house for 7 weeks before the start of the study. Animals were dosed every second day (e.o.d.) between 1-3 pm subcutaneously with a dose volume of 5 ml/kg over a period of 6 days.

Groups of Animals were dosed as follows:

(a) were given the DMS7620 at 3 mg/kg (DMS7620 3 mg/kg GROUP)

(b) were given the DMS7620 at 1 mg/kg (DMS7620 1 mg/kg GROUP)

(c) were given the DMS7620 at 0.3 mg/kg (DMS7620 0.3 mg/kg GROUP)

(d) were given the DMS7620 at 0.1 mg/kg (DMS7620 0.1 mg/kg GROUP)

(e) were given vehicle (Citrate Buffer: 20 mM citrate and 100 mM NaCl)

Note that the animals were also dosed at 0.03 mg/kg, 0.01 mg/kg and 0.003 mg/kg. But these doses were below the threshold for efficacy in this study.

A one day vehicle lead in period was used before the start of drug. Body weight measurements were taken frequently starting four days before the first drug dose, with the first measurement being used to randomize the animals. Food hopper weights were measured frequently starting four days before the first drug dose, allowing for the calculation of food intake. Animals that created excessive food spillage were removed prior to the beginning of the study. During the study, excess food was removed from the cage and added to the food hopper weights for increased accuracy. Five animals (n=5) per group were used for all groups.

Results for example 10 are shown below in Table 6.

### A) Effect of PYY3-36 AlbudAb (DMS7620) on Body Weight

Multiple doses of the PYY3-36 AlbudAb (DMS7620) demonstrated significant decreases in body weight. The day 6 percent change in body weight was 0.0% for vehicle control, −10.4% for DMS7620 (3 mg/kg), −4.6% for DMS7620 (1 mg/kg), −1.7% for DMS7620 (0.3 mg/kg), and −2.2% for DMS7620 (0.1 mg/kg). The 3.0 mg/kg, 1.0 mg/kg, and 0.3 mg/kg doses of DMS7620 were significantly different than vehicle controls.

### B) Effect of PYY3-36 AlbudAb (DMS7620) on Food Intake

Significant inhibition of food intake was observed for the 3.0 mg/kg, 1.0 mg/kg, and 0.3 mg/kg doses of DMS7620 relative to vehicle controls. The average daily food intake over the course of the study was 3.09 grams for vehicle control, 1.52 grams for DMS7620 (3 mg/kg), 2.34 grams for DMS7620 (1 mg/kg), 2.64 grams for DMS7620 (0.3 mg/kg), and 2.76 grams for DMS7620 (0.1 mg/kg). This corresponds to a 51.2% decrease in food intake for the DMS7620 (3 mg/kg), a 20.8% decrease for DMS7620 (1 mg/kg), an 11.8% decrease for DMS7620 (0.3 mg/kg), and a 16.6% decrease for DMS7620 (0.1 mg/kg).

TABLE 6

| | Δ BW (%) | SEM | Ave FI (g) | SEM |
|---|---|---|---|---|
| Vehicle | 0.0% | 0.56% | 3.09 | 0.07 |
| DMS7620 (3 mg/kg) | −10.4%** | 1.75% | 1.52** | 0.19 |
| DMS7620 (1 mg/kg) | −4.6%** | 0.74% | 2.34** | 0.08 |
| DMS7620 (0.3 mg/kg) | −1.7%* | 0.51% | 2.64* | 0.12 |
| DMS7620 (0.1 mg/kg) | −2.2% | 0.81% | 2.76 | 0.12 |

*p < 0.05 vs vehicle;
**p < 0.01 vs vehicle
BW = Body Weight
FI = Food Intake
Ave FI (g) = Average daily food intake for the study duration in grams

### Example 11

### Single AlbudAb Fusions were Made with Both Exendin-4 and Peptide YY

DAT0116 was cloned into the mammalian expression vector pTT5 with an N terminal secretion signal and a C terminal cysteine was introduced using extension of mutagenic oligos and DPNI digestion of template DNA (Stratagene Quickchange). The DNA was sequence verified and transiently transfected into HEK293 cells.

Mammalian cell supernatants were clarified and purified using Protein L affinity chromatography and protein mass was confirmed by mass spectrometry. Proteins were removed from storage at 4 degrees and DAT0116R108C was concentrated in 2×20ml concentrators to 12.5m1. DTT was added to final concentration 5 mM and samples were incubated for 15 minutes. Proteins were then desalted into 20 mM Bis Tris, pH6.57, 5 mM EDTA, 10% Glycerol. Desalted fractions were pooled and for the R108C derivatives 1/10^{th} volume (approx. 2 mgs) was added to 50 ml falcon tubes containing n-ethylmaleimide. The remaining pooled protein was added to various masses of PYY peptide (batch ‘190’) in 50 ml falcons. The samples were incubated rolling at room temperature for 30 minutes, spun for 10 minutes in a bench top centrifuge at 4,500 rpm, analysed by SDS-PAGE and then stored overnight at 4 degrees.

Precipitation was observed in both the R108C derivative coupling reactions with the sample turning opaque shortly after the addition of protein and large flecks forming within 5 minutes. No precipitation was observed in the other reactions.

Post overnight storage the solutions appeared slightly cloudy, however, on standing the cloudiness and pellet were less easy to discern.

Samples were diluted 1/5 with 50 mM Sodium Acetate, pH4.5 and applied to 2×6 ml Resource S columns (previously cleaned with 0.5 M NaOH and equilibrated with dilution buffer) at 2.5 ml/min. Post samples application the column was washed with dilution buffer and then subjected to a 0-100% gradient with 50 mM Sodium Acetate, pH4.5, 1 M NaCl. The column was then washed with 2XPBS and finally cleaned with 0.5 M NaOH.

The Sodium Acetate fractions and the 2×PBS fractions were concentrated separately in multiple 20 ml centrifugal concentrators, analysed by SDS-PAGE, filter sterilized and dialysed against 2×2 L Sodium Citrate, pH6, 100 mM NaCl. The proteins were submitted for MS analysis.

Due to slight contamination of the DAT0116R108C:190PYY with peptide these proteins and the corresponding Sodium Acetate fraction pools were reapplied to a Protein L column.

A 1 ml Protein L column was equilibrated with 1×PBS and cleaned with 6 M Guanidine HCl. The column was re-equilibrated with 1×PBS at 2 ml/min and the DAT0115R108C:190 PYY Sodium Acetate elution pool was applied. Post application the column was washed with 100 mM Sodium Citrate, pH6 and finally eluted with 100 mM Citric acid with a pH of 2.6. The column was re-equilibrated with 100 mM Sodium Citrate, pH6 and the 2XPBS elution pool was applied and purified in a similar manner. The column was cleaned with 6 M Guanidine HCl and the process was repeated for the DAT0116R108C:190 PYY derivatives. The proteins were concentrated to between 1-1.5 ml and were dialysed into 1.6 L 50 mM Sodium Acetate, pH6, 100 mM NaCl overnight at room temperature. The following morning the proteins were withdrawn from the dialysis cassettes, the OD measured, 200 ul concentrated to 20 ul for SDS-PAGE analysis.

Samples of the Exendin-4 AlbudAb peptide YY constructs were submitted for Y2 receptor assay to determine the function of the peptide YY and for GLP-1 receptor assay to determine the function of the Exendin-4. Table 10 shows the activity for Exendin-4 AlbudAb blocked with n-ethyl maleimide (DAT0116 nEM) and Exendin-4 AlbudAb modified with peptide YY (DAT0116 R108C 190PYY). The peptide YY modified Exendin-4 AlbudAb fusion shows a decrease in activity at the Y2 receptor over the peptide control and similar potency at the GLP-1 receptor. The PYY peptide is included as a control. Results are shown in Table 7.

TABLE 7

| | Mean | | DAT01 |
|---|---|---|---|
| Type | pEC50 | Stdev | EC50(pM) |
| DAT0116 R108C NEM | 6.86 | 0 | 1219 |
| DAT0116 R108C 190PYY | 7.33 | 0 | 770 |
| PYY3-36-Mal 190 | 8.51 | 0.15 | N/D |
| PYY3-36-Mal 190 | 8.42 | 0.26 | N/D |

### Example 12

### Expression of DOM7h-14-10 AlbudAb and PYY Genetic Fusion

PYY 3-36 with an additional glycine introduced at the C-terminal, was cloned as a fusion with DOM7h-14-10 (a domain antibody (dAb) which binds serum albumin (albudab) with an amino acid sequence shown below) into the pET30a vector (obtainable from Novagen (Merck)). The PYY was at the 3′ end of the construct and the dAb at the 5′ end. A TVAAPS linker was also introduced between the dAb and PYY sequence; the linker was included as a spacer to separate the dAb spatially from the PYY to prevent steric hindrance of the binding between the PYY and the NP receptor. The amino acid sequence of this construct is shown below and in FIG. 1 (v), SEQ ID NO 49:

(SEQ ID NO 49)
MDIQMTQSPSSLSASVGDRVTITCRASQWIGSQLSWYQQKPGKAPKLLIM
WRSSLQSGVPSRFSGSGSGTDFTLTISSLQPEDFATYYCAQGLRHPKTFG
QGTKVEIKRTVAAPSIKPEAPGEDASPEELNRYYASLRHYLNLVTRQRYG.

Plasmid DNA was prepared in E. coli using alkaline lysis (using a miniprep kit, obtainable from Qiagen CA) and used to transform BL21(DE3) cells (obtainable from Invitrogen). A singly colony was picked and grown overnight at 37° C. in 100 ml of TB media at and then used to inoculate a 1 L culture via a 1/100 dilution. This culture was grown until the OD reached 0.7, at which point protein expression was induced by the addition of IPTG to a final concentration of 70 μM. The culture was grown overnight at 23° C. then harvested by centrifugation and the pellet was stored at −20° C. Thereafter inclusion bodies were prepared by lysing the cells with Bugbuster mix (12.5 ml 10× bugbuster (Merck), 112.5 ml PBS, 250 μl lysonase (Merck) and 4 complete protease inhibitor tablets (Roche). A pellet derived from 500 ml culture was resuspended in 100 ml bugbuster mix and incubated at room temperature for 30 minutes with agitiation then centrifuged at 32000 g for 20 minutes, and the supernatant was discarded. The pellet was washed in 2 M urea in PBS then centrifuged at 32000 g for 15 minutes and the supernatant was discarded. The pellet was then resuspended in 1/12.5 of the original culture volume of 8 M urea in buffer B (100 mM NaCl, 100 mM Tris-HCl pH 8.0, 5% glycerol), agitated at room temperature for 1 hour and then centrifuged at 16000 rpm for 15 minutes. The supernatant (inclusion body prep) was stored at 4° C.

Protein was refolded by dilution by 1/50 into refolding buffer (100 mM MES pH 6.0, 60 mM NaCl, 0.001% triton-X100), filtered and then concentrated. Where required amidation at the C-terminal was achieved by incubating the refolded protein at 8 μM at room temperature over night with 100 mM MES pH 6.0, 0.001% Triton X-100, 30 mM NaCl, 1% Ethanol, 10 μg/mlcatalase, 2.5 mM sodium ascorbate, 1 μM copper chloride and 80 nM peptidylglycine alpha-amidating monooxygenase. Amidation was confirmed by mass spectrometry analysis (MW of glycine-extended fusion protein=16592; MW of C-terminal amidated fusion protein=16534).

Purification was performed on a HiTrap SPFF cation exchange column equilibrated into buffer Y and eluted over a 0-100% gradient of buffer Z. Buffer Y =20 mM sodium citrate pH 5.0; buffer Z=20 mM sodium citrate pH 5.0+1 M NaCl. Thereafter protein was buffer-exchanged into 20 mM sodium citrate pH 6.2 plus 100 mM NaCl, concentrated and stored at −80° C.

1-46. (canceled)

47. A composition which comprises a single fusion or conjugate, wherein said fusion or conjugate comprises or consists of (a) at least two molecules which are selected from insulinotropic and/or incretin and/or gut peptide molecules and which are present as a fusion or conjugate with (b) a protein or peptide which extends the half life of the insulinotropic and/or incretin and/or gut peptide molecule and wherein said protein or peptide which extends half life comprises a domain antibody (dAb) which binds specifically to serum albumin, for example human serum albumin.

48. A composition, which comprises at least two individual fusions or conjugates and wherein each individual fusion or conjugate comprises or consists of (a) one or more molecules selected from: insulinotropic and/or incretin and/or gut peptide molecules; present as a fusion or as a conjugate with (b) a protein or peptide which extends the half life of the insulinotropic and/or incretin and/or gut peptide molecules and wherein said protein or peptide which extends half life comprises a domain antibody (dAb) which binds specifically to serum albumin, for example human serum albumin.

49. A composition according to claim 47, wherein at least one of the insulinotropic and/or incretins is selected from: a GLP-1, PYY, exendin; or a peptide which is a functional variant, analogue, mutant or derivative thereof which retains insulinotropic and/or incretin activity.

50. A composition according to claim 47, wherein at least one of the incretins is selected from: (a) the GLP-1 (7-37) A8G mutant which has the amino acid sequence shown in FIG. 1 (i) (SEQ ID NO 9) or a mutant, derivative or analogue thereof, (b) the exendin-4 molecule which has the amino acid sequence shown in FIG. 1 (j) (SEQ ID NO 10) or a mutant, derivative or analogue thereof; and (c) a PYY peptide which has the amino acid sequence shown in FIG. 1 (s) (SEQ ID NO 19) or a mutant derivative or analogue thereof.

51. A composition according to claim 47, wherein the domain antibody (dAb) which binds specifically to serum albumin is selected from: the DOM 7h-14 (Vk) domain antibody (dAb), (the amino acid sequence of DOM 7h-14 is shown in FIG. 1(h): SEQ ID NO 8), or the DOM 7h-14 -10(Vk) domain antibody (dAb), (the amino acid sequence of DOM 7h-14-10 is shown in FIG. 1(o): SEQ ID NO 15), and the DOM 7h-14 -10(Vk) domain antibody (dAb) which has the R108C mutation (the amino acid sequence of DOM 7h-14-10 R108 C is shown in FIG. 1(r) SEQ ID NO 18); and the 7h-11-15 albudab (the amino acid sequence of DOM 7h-11-15 is shown in FIG. 1(p): SEQ ID NO 16) and the 7h-11-15 R108 C albudab (the amino acid sequence of DOM 7h-11-15 R108 C is shown in FIG. 1(T): SEQ ID NO 47); or a dAb which binds to the same epitope on serum albumin or which competes with any of these for binding to serum albumin.

52. A composition according to claim 47, which further comprises an amino acid or chemical linker joining the insulinotropic and/or incretin molecule and/or gut peptide and the dAb that binds to serum albumin e.g. wherein the amino acid linker is selected from: a helical linker with the amino acid sequence shown in FIG. 1 (k) (SEQ ID NO 11), the gly-ser linker with the amino acid sequence shown in FIG. 1 (l) (SEQ ID NO 12), or a PEG linker such as the PEG linker which has the structure of the PEG linker shown in FIG. 3.

53. A composition according to claim 47, which comprises one or more of the peptide-AlbudAb molecules specified in: FIGS. 1a-1g (SEQ ID NOS 1-7); and FIGS. 1m-1n (SEQ ID NOS 13-14); and FIGS. 1u-v (SEQ ID NOS 48-49); and FIG. 3 or the Dom7h-11-15 (R108C)-PEG-3-36 PYY (Lysine at position 10) (with the structure shown in FIG. 3 except that the albudab component is the Dom7h-11-15 (R108C).

54. A composition according to claim 47, which comprises (a) the DAT0115 molecule (with the amino acid sequence shown in FIG 1b: SEQ ID NO 2) and either (b) the Dom7h-14-10 (R108C)-PEG-3-36 PYY (Lysine at position 10) (with the structure shown in FIG. 3) as a combined preparation for simultaneous, separate or sequential use or (c) the Dom7h-11-15 (R108C)-PEG-3-36 PYY (Lysine at position 10) (with the structure shown in FIG. 3 except that the albudab component is the Dom7h-11-15 (R108C).

55. A composition according to claim 47, wherein the fusion or conjugate binds to human serum albumin with KD in the range of about 5 micromolar to about 1 picomolar.

56. A pharmaceutical composition which comprises a composition according to claim 47 in combination with a pharmaceutically or physiologically acceptable carrier, excipient or diluent.

57. A composition which comprises (a) a composition according to claim 47 and which comprises (b) further therapeutic or active agents; for separate, sequential or concurrent administration to a subject.

58. A composition which comprises the two or more fusions or conjugates of claim 48, which each comprise or consist of (a) one or more insulinotropic and/or incretin and/or gut peptide molecules, present as a fusion or conjugate with (b) a domain antibody (dAb) which binds specifically to serum albumin, as a combined preparation for simultaneous, separate or sequential use in therapy.

59. A composition according to claim 47, for use in treating or preventing a metabolic disease or disorder e.g. wherein the disease or disorder is selected from:
hyperglycemia, impaired glucose tolerance, beta cell deficiency, diabetes (type 1 or type 2 diabetes or gestational diabetes), obesity, diseases characterised by overeating.

60. An oral, injectable, inhalable or nebulisable formulation which comprises a composition according to claim 47.

61. An isolated or recombinant nucleic acid encoding a fusion as referenced in claims 47 to 59.


SEQUENCE LISTING (49 sequences)

SEQ ID NO: 1 - protein, 168 aa, Artificial Sequence
  2xGLP-1 A8G DOM7h-14 fusion (DAT0114)
HGEGTFTSDVSSYLEGQAAKEFIAWLVKGRHGEGTFTSDVSSYLEGQAAKEFIAWLVKGRDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 2 - protein, 163 aa, Artificial Sequence
  Exendin 4, (G4S)3 linker, DOM7h-14 fusion (DAT0115)
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 3 - protein, 148 aa, Artificial Sequence
  Exendin 4 DOM7h-14 fusion (DAT0116)
HGEGTFTSDLSKQMEEEAVRLFIEWLKNGGPSSGAPPPSGDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 4 - protein, 188 aa, Artificial Sequence
  Exendin 4, helical linker, DOM7h-14 fusion (DAT0117)
HGEGTFTSDLSKQMEEEAVRLFIEWLKNGGPSSGAPPPSGKEAAAKEAAAKEAAAKELAAKEAAAKEAAAKEAAAKELAADIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 5 - protein, 153 aa, Artificial Sequence
  GLP-1 A8G, (G4S)3, linker DOM7h-14 fusion (DAT0118)
HGEGTFTSDVSSYLEGQAAKEFIAWLVKGRGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 6 - protein, 142 aa, Artificial Sequence
  GLP-1 A8G, PSS linker, DOM7h-14 fusion (DAT0119)
HGEGTFTSDVSSYLEGQAAKEFIAWLVKGRGPSSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 7 - protein, 179 aa, Artificial Sequence
  GLP-1 A8G, helical linker, DOM7h-14 fusion (DAT0120)
HGEGTFTSDVSSYLEGQAAKEFIAWLVKGRGKEAAAKEAAAKEAAAKELAAKEAAAKEAAAKEAAAKELAADIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 8 - protein, 108 aa, Artificial Sequence
  DOM7h-14:
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 9 - protein, 31 aa, Artificial Sequence
  GLP-1 (7-37) A8G:
HGEGTFTSDVSSYLEGQAAKEFIAWLVKGRG

SEQ ID NO: 10 - protein, 39 aa, Artificial Sequence
  exendin-4:
HGEGTFTSDLSKQMEEEAVRLFIEWLKNGGPSSGAPPPS

SEQ ID NO: 11 - protein, 40 aa, Artificial Sequence
  helical linker
KEAAAKEAAAKEAAAKELAAKEAAAKEAAAKEAAAKELAA

SEQ ID NO: 12 - protein, 16 aa, Artificial Sequence
  Gly-ser linker:
GGGGGSGGGGSGGGGS

SEQ ID NO: 13 - protein, 163 aa, Artificial Sequence
  Exendin 4, (G4S)3, linker DOM7h-14-10 fusion (DMS7139)
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 14 - protein, 163 aa, Artificial Sequence
  Exendin 4, (G4S)3, linker DOM7h-11-15 fusion (DMS7143)
HGEGTFTSDLSKQMEEEAVRLFIEWLKNGGPSSGAPPPSGGGGGSGGGGSGGGGSDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 15 - protein, 108 aa, Artificial Sequence
  DOM7h-14-10
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 16 - protein, 108 aa, Artificial Sequence
  DOM7h-11-15
DIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 17 - protein, 20 aa, Artificial Sequence
  OmpT AWA signal peptide (leader)
MRAKLLGIVLTTPIAISAWA

SEQ ID NO: 18 - skipped

SEQ ID NO: 19 - protein, 34 aa, Artificial Sequence
  PYY 3-36 (with a lysine at position 10)
IKPEAPGKDASPEELNRYYASLRHYLNLVTRQRY

SEQ ID NO: 20 - DNA, 504 bp, Artificial Sequence
  DAT0114 - nucleic acid sequence (from mammalian construct):
catggtgaagggacctttaccagtgatgtaagttcttatttggaaggccaagctgccaaggaattcattgcttggctggtgaaaggccgacatggtgaagggacctttaccagtgatgtaagttcttatttggaaggccaagctgccaaggaattcattgcttggctggtgaaaggccgagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 21 - DNA, 489 bp, Artificial Sequence
  DAT0115 - nucleic acid sequence (from mammalian construct):
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 22 - DNA, 495 bp, Artificial Sequence
  DAT0115 - nucleic acid sequence (from E.coli construct):
cacggtgaaggtaccttcacctctgacctgagcaaacagatggaggaagaagcggttcgtctgttcatcgagtggctgaaaaacggtggtccgtcttctggtgctccgccaccgtctggtggtggtggtggttctggtggtggtggttctggtggtggcggtagcgacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 23 - DNA, 444 bp, Artificial Sequence
  DAT0116 - nucleic acid sequence (from mammalian construct):
catggtgaaggaacatttaccagtgacttgtcaaaacagatggaagaggaggcagtgcggttatttattgagtggcttaagaacggaggaccaagtagcggggcacctccgccatcgggtgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 24 - DNA, 447 bp, Artificial Sequence
  DAT0116 - nucleic acid sequence (from E.coli construct):
cacggtgaaggtaccttcacctctgacctgagcaaacagatggaggaagaagcggttcgtctgttcatcgagtggctgaaaaacggtggtccgtcttctggtgctccgccaccgtctgacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 25 - DNA, 564 bp, Artificial Sequence
  DAT0117 - nucleic acid sequence (from mammalian construct):
catggtgaaggaacatttaccagtgacttgtcaaaacagatggaagaggaggcagtgcggttatttattgagtggcttaagaacggaggaccaagtagcggggcacctccgccatcgggtaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 26 - DNA, 567 bp, Artificial Sequence
  DAT0117 - nucleic acid sequence (from E.coli construct):
cacggtgaaggtaccttcacctctgacctgagcaaacagatggaggaagaagcggttcgtctgttcatcgagtggctgaaaaacggtggtccgtcttctggtgctccgccaccgtctaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 27 - DNA, 459 bp, Artificial Sequence
  DAT0118 - nucleic acid sequence (from mammalian construct):
catggtgaagggacctttaccagtgatgtaagttcttatttggaaggccaagctgccaaggaattcattgcttggctggtgaaaggccgaggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 28 - DNA, 426 bp, Artificial Sequence
  DAT0119 - nucleic acid sequence (from mammalian construct):
catggtgaagggacctttaccagtgatgtaagttcttatttggaaggccaagctgccaaggaattcattgcttggctggtgaaaggccgaggaccaagctcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 29 - DNA, 537 bp, Artificial Sequence
  DAT0120 - nucleic acid sequence (from mammalian construct):
catggtgaagggacctttaccagtgatgtaagttcttatttggaaggccaagctgccaaggaattcattgcttggctggtgaaaggccgaggaaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 30 - DNA, 324 bp, Artificial Sequence
  Dom7h-14 - nucleic acid sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 31 - DNA, 489 bp, Artificial Sequence
  Exendin 4, (G4S)3, linker DOM7h-14-10 fusion (DMS7139)
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 32 - DNA, 489 bp, Artificial Sequence
  Exendin 4, (G4S)3, linker DOM7h-11-115 fusion (DMS7143)
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 33 - DNA, 324 bp, Artificial Sequence
  Dom7h-14-10  - nucleic acid
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 34 - DNA, 324 bp, Artificial Sequence
  Dom7h-11-15  - nucleic acid
gacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 35 - DNA, 60 bp, Artificial Sequence
  OmpAWA signal peptide  - nucleic acid sequence
atgcgggcgaaactcctaggaatagtcctgacaacccctatcgcgatcagcgcttgggcc

SEQ ID NO: 36 - DNA, 324 bp, Artificial Sequence
  Dom7h-14-10  R (108) C - nucleic acid sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaatgc

SEQ ID NO: 37 - protein, 142 aa, Artificial Sequence
  Dom7h-14-10 (R108C) albudab conjugated to a C-terminally amidated PYY3-36 via a lysine (introduced at position 10 of PYY)
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKCIKPEAPGKDASPEELNRYYASLRHYLNLVTRQRY

SEQ ID NO: 38 - protein, 21 aa, Artificial Sequence
  ompA  (E. coli derived) leader
MKKTAIAIAVALAGFATVAQA

SEQ ID NO: 39 - protein, 21 aa, Artificial Sequence
  ompA-AMA  (artificial sequence)
MKKTAIAIAVALAGFATVAMA

SEQ ID NO: 40 - protein, 21 aa, Artificial Sequence
  ompA-AWA  (artificial sequence)
MKKTAIAIAVALAGFATVAWA

SEQ ID NO: 41 - protein, 20 aa, Artificial Sequence
  ompT  (E. coli derived)
MRAKLLGIVLTTPIAISSFA

SEQ ID NO: 42 - protein, 20 aa, Artificial Sequence
  ompT-AMA
MRAKLLGIVLTTPIAISAMA

SEQ ID NO: 43 - protein, 22 aa, Artificial Sequence
  GAS  (S. cerevisiae derived)
MLFKSLSKLATAAAFFAGVATA

SEQ ID NO: 44 - protein, 22 aa, Artificial Sequence
  GAS-AMA
MLFKSLSKLATAAAFFAGVAMA

SEQ ID NO: 45 - protein, 22 aa, Artificial Sequence
  GAS-AWA
MLFKSLSKLATAAAFFAGVAWA

SEQ ID NO: 46 - protein, 22 aa, Artificial Sequence
  Pel B (Erwinia carotovora)
MKYLLPTAAAGLLLLAAQPAMA

SEQ ID NO: 47 - protein, 108 aa, Artificial Sequence
  DOM7h-11-15 R108C
DIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKC

SEQ ID NO: 48 - protein, 35 aa, Artificial Sequence
  DAT 0116R108C: 190 PYY
HIKPEAPGKDASPEELNRYYASLRHYLNLVTRQRY

SEQ ID NO: 49 - protein, 150 aa, Artificial Sequence
  Genetic fusion of PYY-Dom 7h-14-10 albudab
MDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKRTVAAPSIKPEAPGEDASPEELNRYYASLRHYLNLVTRQRYG